(19) United States
(12) Patent Application Publication
(10) Pub. No.: US 2024/0285248 A1
(43) Pub. Date: Aug. 29, 2024

(54) SYSTEMS AND METHODS FOR PREDICTING BIOCHEMICAL PROGRESSION FREE SURVIVAL IN PROSTATE CANCER PATIENTS

(71) Applicants: Progenics Pharmaceuticals, Inc., N. Billerica, MA (US); EXINI Diagnostics AB, Lund (SE)

(72) Inventors: Karl Vilhelm Sjöstrand, Atlantic Highlands, NJ (US); Aseem Undvall Anand, Queens, NY (US)

(21) Appl. No.: 18/530,831

(22) Filed: Dec. 6, 2023

Related U.S. Application Data
(60) Provisional application No. 63/445,257, filed on Feb. 13, 2023.

(51) Int. Cl. A61B 6/00; A61B 6/03; G06T 7/00; G06T 7/11; G16H 50/30

(52) U.S. Cl. CPC A61B 6/5217; A61B 6/032; A61B 6/037; A61B 6/50; A61B 6/5235; A61B 6/563; G06T 7/0012; G06T 7/11; G16H 50/30; G06T 2207/10081; G06T 2207/10104; G06T 2207/20081; G06T 2207/20084; G06T 2207/30081; G06T 2207/30096

(57) ABSTRACT

Presented herein are systems and methods for predicting biochemical progression free survival (bPFS) in prostate cancer patients. In certain embodiments, bPFS is predicted from ¹⁸F-DCFPyL PET/CT images using deep learning (or other machine learning or artificial intelligence techniques) to segment anatomical information from the CT image and use this information in combination with the PET image to detect and quantify candidates for prostate cancer lesions.

### CROSS REFERENCE TO RELATED APPLICATIONS

This application claims priority to and benefit of U.S. provisional application No. 63/445,257, filed Feb. 13, 2023, the contents of which are incorporated by reference herein in their entirety.

### TECHNICAL FIELD

This invention relates generally to systems and methods for analysis of medical images to identify and/or characterize cancerous lesions, prognosis, and/or risk for a subject.

### SUMMARY

Presented herein are systems and methods for predicting biochemical progression free survival (bPFS) in prostate cancer patients. In certain embodiments, bPFS is predicted from ¹⁸F-DCFPyL PET/CT images using deep learning (or other machine learning or artificial intelligence techniques) to segment anatomical information from the CT image and use this information in combination with the PET image to detect and quantify candidates for prostate cancer lesions.

In one aspect, the invention is directed to a method for processing one or more images of a prostate cancer patient to automatically determine a patient risk index that correlates with biochemical progression free survival (bPFS) in the patient, the method comprising: (a) receiving, by a processor of a computing device, an image of the subject obtained using a functional imaging modality (e.g., a 3D PET/CT image); and (b) identifying, by the processor, one or more patient risk index/indices that correlate with bPFS in the patient. In certain embodiments, the identifying step comprises using deep learning to segment anatomical information from the CT image and use this information in combination with the PET image to detect and quantify candidates for prostate cancer lesions.

In certain embodiments, an image of the subject is or comprises a 3D PET/CT image.

In certain embodiments, an identifying step (e.g., step (b)) comprises using deep learning [e.g., a machine learning module (e.g., a Convolutional Neural Network (CNN))] to segment anatomical information from the CT image and use the anatomical information in combination with the PET image to detect and quantify candidates for prostate cancer lesions.

In certain embodiments, one or more determined patient risk index/indices that correlate with bPFS in the patient include one or more members selected from the group consisting of (i) SUVₘₑₐₙ (SUV=standard update value), (ii) SUVₘₐₓ, (iii) PSMA positive total tumor volume (PSMAₜₜᵥ), and (iv) aPSMA scores.

In certain embodiments, PSMAₜₜᵥ is a measure of total lesion volume (e.g., sum carried out over hospots that are identified as lesions via artificial intelligence and/or other algorithm and/or with user validation, e.g., where the total volume may be subdivided based on the locality of the lesion, e.g., one volume for miT, one for miN, one for miMa, and the like). In certain embodiments, PSMAₜₜᵥ is a measure of a total lesion volume for the subject and/or over a subset of lesions within one or more tissue regions (e.g., organs) and/or prostate cancer staging classes (e.g., miTNM classes) [e.g., a sum carried out over hospots that are identified as lesions via artificial intelligence (e.g., a machine learning module (e.g., a CNN) and/or other algorithm and/or with user validation (e.g., as described in Section C), e.g., where the total volume may be subdivided based on the locality of the lesion, e.g., one volume for miT, one for miN, one for miMa, and the like (e.g., as described in Section E)].

In certain embodiments, an aPSMA score is a quantitative score for tumor burden measuring the interaction of tumor volume and uptake stratified by local tumors (aPSMA-miT), regional lymph nodes (aPSMA-miN) and distant metastases (aPSMA-miMa for extrapelvic metastases, miMb for bone metastases and miMc for other organ metastases). In certain embodiments, the aPSMA score is an intensity-weighted tissue lesion volume (ITLV), e.g., a weighted sum of the lesion volumes of a specific type, weighted by lesion index LI. In certain embodiments, an aPSMA score is a quantitative score for tumor burden measuring the interaction of tumor volume and uptake [e.g., an overall measure, computed over all lesions identified for the subject; e.g., stratified by local tumors (aPSMA-miT), regional lymph nodes (aPSMA-miN) and distant metastases (aPSMA-miMa for extrapelvic metastases, miMb for bone metastases and miMc for other organ metastases)] [e.g., wherein the aPSMA score is an intensity-weighted tissue lesion volume (ITLV), e.g., a weighted sum of the lesion volumes of a specific type, weighted by lesion index LI].

In certain embodiments, step (b) comprises detecting, by the processor, one or more hotspots (e.g., localized regions of elevated intensity relative to their surroundings) within the functional image, each hotspot determined to represent a potential underlying lesion; and determining, by the processor, (e.g., values of) the one or more patient risk indices based on the one or more detected hotspots. In certain embodiments, detecting the one or more hotspots comprises using a deep learning model (e.g., a convolutional neural network).

In certain embodiments, an image of the subject is a nuclear medicine image obtained following administration to the subject of a PSMA binding agent. In certain embodiments, a PSMA binding agent is or comprises [18F]DCFPyL (PyL).

In certain embodiments, the method comprises causing, by the processor, display of the one or more patient risk indices within a graphical user interface (GUI) (e.g., a GUI of a decision support system).

In certain embodiments, a processor is a processor of a cloud-based system.

In another aspect, the invention is directed to a system for processing one or more images of a prostate cancer patient to automatically determine a patient risk index that correlates with biochemical progression free survival (bPFS) in the patient, the system comprising: a processor of a computing device; and memory having instructions stored thereon, wherein the instructions, when executed by the processor, cause the processor to: (a) receive an image of the subject obtained using a functional imaging modality (e.g., a 3D PET/CT image); and (b) identify one or more patient risk index/indices that correlate with bPFS in the patient.

In certain embodiments, an image of the subject is or comprises a 3D PET/CT image.

In certain embodiments, at step (b), the instructions cause the processor to identify the one or more patient risk index/indices by using deep learning [e.g., a machine learning module (e.g., a Convolutional Neural Network (CNN))] to segment anatomical information from the CT image and use the anatomical information in combination with the PET image to detect and quantify candidates for prostate cancer lesions.

In certain embodiments, one or more determined patient risk index/indices that correlate with bPFS in the patient include one or more members selected from the group consisting of (i) SUVₘₑₐₙ (SUV=standard update value), (ii) SUVₘₐₓ, (iii) PSMA positive total tumor volume (PSMAₜₜᵥ), and (iv) aPSMA scores.

In certain embodiments, PSMAₜₜᵥ is a measure of total lesion volume (e.g., sum carried out over hospots that are identified as lesions via artificial intelligence and/or other algorithm and/or with user validation, e.g., where the total volume may be subdivided based on the locality of the lesion, e.g., one volume for miT, one for miN, one for miMa, and the like). In certain embodiments, PSMAₜₜᵥ is a measure of a total lesion volume for the subject and/or over a subset of lesions within one or more tissue regions (e.g., organs) and/or prostate cancer staging classes (e.g., miTNM classes) [e.g., a sum carried out over hospots that are identified as lesions via artificial intelligence (e.g., a machine learning module (e.g., a CNN) and/or other algorithm and/or with user validation (e.g., as described in Section C), e.g., where the total volume may be subdivided based on the locality of the lesion, e.g., one volume for miT, one for miN, one for miMa, and the like (e.g., as described in Section E)].

In certain embodiments, an aPSMA score is a quantitative score for tumor burden measuring the interaction of tumor volume and uptake stratified by local tumors (aPSMA-miT), regional lymph nodes (aPSMA-miN) and distant metastases (aPSMA-miMa for extrapelvic metastases, miMb for bone metastases and miMc for other organ metastases). In certain embodiments, the aPSMA score is an intensity-weighted tissue lesion volume (ITLV), e.g., a weighted sum of the lesion volumes of a specific type, weighted by lesion index LI. In certain embodiments, an aPSMA score is a quantitative score for tumor burden measuring the interaction of tumor volume and uptake [e.g., an overall measure, computed over all lesions identified for the subject; e.g., stratified by local tumors (aPSMA-miT), regional lymph nodes (aPSMA-miN) and distant metastases (aPSMA-miMa for extrapelvic metastases, miMb for bone metastases and miMc for other organ metastases)] [e.g., wherein the aPSMA score is an intensity-weighted tissue lesion volume (ITLV), e.g., a weighted sum of the lesion volumes of a specific type, weighted by lesion index LI].

In certain embodiments, at step (b), the instructions cause the processor to detect one or more hotspots (e.g., localized regions of elevated intensity relative to their surroundings) within the functional image, each hotspot determined to represent a potential underlying lesion; and determine (e.g., values of) the one or more patient risk indices based on the one or more detected hotspots.

In certain embodiments, the instructions cause the processor to detect the one or more hotspots comprises using a deep learning model (e.g., a convolutional neural network).

In certain embodiments, an image of the subject is a nuclear medicine image obtained following administration to the subject of a PSMA binding agent. In certain embodiments, a PSMA binding agent is or comprises [18F]DCFPyL (PyL).

In certain embodiments, the instructions cause the processor to cause display of the one or more patient risk indices within a graphical user interface (GUI) (e.g., a GUI of a decision support system).

In certain embodiments, the system is a cloud-based system.

Features of embodiments described with respect to one aspect of the invention may be applied with respect to another aspect of the invention.

### BRIEF DESCRIPTION OF THE DRAWING

The foregoing and other objects, aspects, features, and advantages of the present disclosure will become more apparent and better understood by referring to the following description taken in conjunction with the accompanying drawings, in which:

FIG. 1A is a set of three images showing corresponding slices of a CT image, a PET image, and a PET/CT fusion, obtained from a 3D PET/CT scan, according to an illustrative embodiment.

FIG. 1B is an image showing a set of two slices of a PET/CT composite image in which a PET image is overlaid on a CT scan, according to an illustrative embodiment.

FIG. 2 is a diagram illustrating an example process for segmenting an anatomical image and identifying anatomical boundaries in a co-aligned functional image, according to an illustrative embodiment.

FIG. 3 is a diagram illustrating an example process for segmenting and classifying hotspots, according to an illustrative embodiment.

FIG. 4A is a screenshot of a graphical user interface (GUI) showing a computer-generated report for a patient via image analysis and decision support tools of the present disclosure, according to an illustrative embodiment.

FIG. 4B is a schematic showing an approach for computing lesion index values, according to an illustrative embodiment.

FIG. 5A is a block flow diagram of an example process for identifying bPFS-correlated patient risk indices, according to an illustrative embodiment.

FIG. 5B is a block flow diagram of an example process for determining and reporting bPFS-correlated patient risk indices, according to an illustrative embodiment.

FIG. 6 is a block diagram of an exemplary cloud computing environment, used in certain embodiments.

FIG. 7 is a block diagram of an example computing device and an example mobile computing device used in certain embodiments.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### CERTAIN DEFINITIONS

In order for the present disclosure to be more readily understood, certain terms are first defined below. Additional definitions for the following terms and other terms are set forth throughout the specification.

A, an: The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

About, approximately: As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

First, second, etc.: It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed or that the first element must precede the second element in some manner. In addition, unless stated otherwise, a set of elements may comprise one or more elements.

Image: As used herein, an “image”—for example, a 3D image of subject, includes any visual representation, such as a photo, a video frame, streaming video, as well as any electronic, digital or mathematical analogue of a photo (e.g., a digital image), video frame, or streaming video, displayed or stored in memory (e.g., a digital image may, but need not be displayed for visual inspection). Any apparatus described herein, in certain embodiments, includes a display for displaying an image or any other result produced by the processor. Any method described herein, in certain embodiments, includes a step of displaying an image or any other result produced via the method. In certain embodiments, an image is a 3D image, conveying information that varies with position within a 3D volume. Such images may, for example, be represented digitally as a 3D matrix (e.g., a N×M×L matrix) with each voxel of a 3D image represented by an element of a 3D matrix. Other representations are also contemplated and included, for example, a 3D matrix may be reshaped as a vector (e.g., a 1×K size vector, where K is a total number of voxels) by stitching each row or column end to end. Examples of images include, for example, medical images, such as bone-scan images (also referred to as scintigraphy images), computed tomography (CT) images, magnetic resonance images (MRIs), optical images (e.g., bright-field microscopy images, fluorescence images, reflection or transmission images, etc.), positron emission tomography (PET) images, single-photon emission tomography (SPECT) images, ultrasound images, x-ray images, and the like. In certain embodiments, a medical image is or comprises a nuclear medicine image, produced from radiation emitted from within a subject being imaged. In certain embodiments, a medical image is or comprises an anatomical image (e.g., a 3D anatomical image) conveying information regarding location and extent of anatomical structures such as internal organs, bones, soft-tissue, and blood vessels, within a subject. Examples of anatomical images include, without limitation, x-ray images, CT images, MRIs, and ultrasound images. In certain embodiments, a medical image is or comprises a functional image (e.g., a 3D functional image) conveying information relating to physiological activities within specific organs and/or tissue, such as metabolism, blood flow, regional chemical composition, absorption, etc. Examples of functional images include, without limitation, nuclear medicine images, such as PET images, SPECT images, as well as other functional imaging modalities, such as functional MRI (fMRI), which measures small changes in blood flow for use in assessing brain activity.

Map: As used herein, the term “map” is understood to mean a visual display, or any data representation that may be interpreted for visual display, which contains spatially-correlated information. For example, a three-dimensional map of a given volume may include a dataset of values of a given quantity that varies in three spatial dimensions throughout the volume. A three-dimensional map may be displayed in two-dimensions (e.g., on a two-dimensional screen, or on a two-dimensional printout).

Segmentation map: As used herein, the term “segmentation map” refers to a computer representation that identifies one or more 2D or 3D regions determined by segmenting an image. In certain embodiments, a segmentation map distinguishably identifies multiple different (e.g., segmented) regions, allowing them to be individually and distinguishably accessed and operated upon and/or used for operating on, for example, one or more images.

3D, three-dimensional: As used herein, “3D” or “three-dimensional” with reference to an “image” means conveying information about three dimensions. A 3D image may be rendered as a dataset in three dimensions and/or may be displayed as a set of two-dimensional representations, or as a three-dimensional representation. In certain embodiments, a 3D image is represented as voxel (e.g., volumetric pixel) data.

Whole body: As used herein, the terms “full body” and “whole body” used (interchangeably) in the context of segmentation and other manners of identification of regions within an image of a subject refer to approaches that evaluate a majority (e.g., greater than 50%) of a graphical representation of a subject's body in a 3D anatomical image to identify target tissue regions of interest. In certain embodiments, full body and whole body segmentation refers to identification of target tissue regions within at least an entire torso of a subject. In certain embodiments, portions of limbs are also included, along with a head of the subject.

Radionuclide: As used herein, “radionuclide” refers to a moiety comprising a radioactive isotope of at least one element. Exemplary suitable radionuclides include but are not limited to those described herein. In some embodiments, a radionuclide is one used in positron emission tomography (PET). In some embodiments, a radionuclide is one used in single-photon emission computed tomography (SPECT). In some embodiments, a non-limiting list of radionuclides includes ^{99m}Tc, ¹¹¹In, ⁶⁴Cu, ⁶⁷Ga, ⁶⁸Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹²⁶I, ¹³¹ I, ¹¹C, ¹³N, ¹⁵O, ¹⁸F, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, ⁸²Rb, ⁷⁵Br, ⁷⁶Br, ⁷⁷Br, ⁸⁰Br, ^{80m}Br, ⁸²Br, ⁸³Br, ²¹¹At and ¹⁹²Ir.

Radiopharmaceutical: As used herein, the term “radiopharmaceutical” refers to a compound comprising a radionuclide. In certain embodiments, radiopharmaceuticals are used for diagnostic and/or therapeutic purposes. In certain embodiments, radiopharmaceuticals include small molecules that are labeled with one or more radionuclide(s), antibodies that are labeled with one or more radionuclide(s), and antigen-binding portions of antibodies that are labeled with one or more radionuclide(s).

Machine learning module: Certain embodiments described herein make use of (e.g., include) software instructions that include one or more machine learning module(s), also referred to herein as artificial intelligence software. As used herein, the term “machine learning module” refers to a computer implemented process (e.g., function) that implements one or more specific machine learning algorithms in order to determine, for a given input (such as an image (e.g., a 2D image; e.g., a 3D image), dataset, and the like) one or more output values. For example, a machine learning module may receive as input a 3D image of a subject (e.g., a CT image; e.g., an MRI), and for each voxel of the image, determine a value that represents a likelihood that the voxel lies within a region of the 3D image that corresponds to a representation of a particular organ or tissue of the subject. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of a CNN module may be carried out via specialized hardware (e.g., via an application specific integrated circuit (ASIC)).

Subject: As used herein, a “subject” means a human or other mammal (e.g., rodent (mouse, rat, hamster), pig, cat, dog, horse, primate, rabbit, and the like).

Administering: As used herein, “administering” an agent means introducing a substance (e.g., an imaging agent) into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments.

Tissue: As used herein, the term “tissue” refers to bone (osseous tissue) as well as soft-tissue.

### DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

As described in further detail herein, and, for example, in PCT/EP2021/068337, filed on Jul. 2, 2021, entitled, “SYSTEMS AND METHODS FOR ARTIFICIAL INTELLIGENCE-BASED IMAGE ANALYSIS FOR DETECTION AN) CHARACTERIZATION OF LESIONS,” and published on Jan. 13, 2022, as International (PCT) Publication No. WO/2022/008374; and (ii) U.S. application Ser. No. 18/207,246, filed Jul. 8, 2023, entitled, “SYSTEMS AND METHODS FOR ASSESSING DISEASE BURDEN AND PROGRESSION,” the content of each of which are incorporated by reference herein in their entirety, in certain embodiments, automated (including semi-automated) analysis of three dimensional (3D) medical images can be used to compute values of patient indices that correlate with disease burden and/or prognosis for a subject. These 3D medical images may include, for example, positron emission tomography (PET) images, single-photon emission computerized tomography (SPECT) images, whole-body bone images, combined PET/CT images (CT=computed tomography), and/or combined SPECT/CT images. The determined patient indices include risk indices that correlate with patient overall survival and other prognostic metrics indicative of disease state, progression, and/or treatment efficacy.

In certain embodiments, a PET image of a patient is obtained using the prostate-specific membrane antigen (PSMA) binding agent PyL™ (also referred to as ¹⁸F-DCFPyL, [18F]DCFPyL, or DCFPyL-18F, with chemical structure as shown in the above-referenced WO/2022/008374) and is overlaid (or otherwise used in combination) with a CT image of the patient. Examples of the automated analysis of these composite ¹⁸F-DCFPvL PET/CT images are described in the above-referenced WO/2022/008374 and U.S. application Ser. No. 18/207,246.

Further to the above, presented herein are systems and methods for predicting biochemical progression free survival (bPFS) in prostate cancer patients. In certain embodiments, bPFS is predicted using ¹⁸F-DCFPyL PET/CT images, though PSMA binding agents other than ¹⁸F-DCFPyL may also be used. Moreover, in certain embodiments, SPECT/CT images may be used in place of PET/CT images. In certain embodiments, the images are analyzed using deep learning (or other machine learning or artificial intelligence techniques) to segment anatomical information from the CT image and use this information in combination with the PET image to detect and quantify candidates for prostate cancer lesions.

It is contemplated that systems, devices, methods, and processes of the claimed invention encompass variations and adaptations developed using information from the embodiments described herein.

Throughout the description, where articles, devices, and systems are described as having, including, or comprising specific components, or where processes and methods are described as having, including, or comprising specific steps, it is contemplated that, additionally, there are articles, devices, and systems of the present invention that consist essentially of, or consist of, the recited components, and that there are processes and methods according to the present invention that consist essentially of, or consist of, the recited processing steps. It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein.

Headers are provided for the convenience of the reader—the presence and/or placement of a header is not intended to limit the scope of the subject matter described herein.

### A. Nuclear Medicine Images

Nuclear medicine images may be obtained using a nuclear medicine imaging modality such as bone scan imaging (also referred to as scintigraphy), Positron Emission Tomography (PET) imaging, and Single-Photon Emission Tomography (SPECT) imaging.

In certain embodiments, nuclear medicine images are obtained using imaging agents comprising radiopharmaceuticals. Nuclear medicine images may be obtained following administration of a radiopharmaceutical to a patient (e.g., a human subject), and provide information regarding the distribution of the radiopharmaceutical within the patient.

Nuclear medicine imaging techniques detect radiation emitted from the radionuclides of radiopharmaceuticals to form an image. The distribution of a particular radiopharmaceutical within a patient may be influenced and/or dictated by biological mechanisms such as blood flow or perfusion, as well as by specific enzymatic or receptor binding interactions. Different radiopharmaceuticals may be designed to take advantage of different biological mechanisms and/or particular specific enzymatic or receptor binding interactions and thus, when administered to a patient, selectively concentrate within particular types of tissue and/or regions within the patient. Greater amounts of radiation are emitted from regions within the patient that have higher concentrations of radiopharmaceutical than other regions, such that these regions appear brighter in nuclear medicine images. Accordingly, intensity variations within a nuclear medicine image can be used to map the distribution of radiopharmaceutical within the patient. This mapped distribution of radiopharmaceutical within the patient can be used to, for example, infer the presence of cancerous tissue within various regions of the patient's body. In certain embodiments, intensities of voxels of a nuclear medicine image, for example a PET image, represent standard uptake values (SUVs) (e.g., having been calibrated for injected radiopharmaceutical dose and/or patient weight).

For example, upon administration to a patient, technetium 99m methylenediphosphonate (^{99m}Tc MDP) selectively accumulates within the skeletal region of the patient, in particular at sites with abnormal osteogenesis associated with malignant bone lesions. The selective concentration of radiopharmaceutical at these sites produces identifiable hotspots—localized regions of high intensity—in nuclear medicine images. Accordingly, presence of malignant bone lesions associated with metastatic prostate cancer can be inferred by identifying such hotspots within a whole-body scan of the patient. In certain embodiments, analyzing intensity variations in whole-body scans obtained following administration of ^{99m}Tc MDP to a patient, such as by detecting and evaluating features of hotspots, can be used to compute, risk indices that correlate with patient overall survival and other prognostic metrics indicative of disease state, progression, treatment efficacy, and the like. In certain embodiments, other radiopharmaceuticals can also be used in a similar fashion to ^{99m}Tc MDP.

In certain embodiments, the particular radiopharmaceutical used depends on the particular nuclear medicine imaging modality used. For example, 18F sodium fluoride (NaF) also accumulates in bone lesions, similar to ^{99m}Tc MDP, but can be used with PET imaging. In certain embodiments, PET imaging may also utilize a radioactive form of the vitamin choline, which is readily absorbed by prostate cancer cells.

In certain embodiments, radiopharmaceuticals that selectively bind to particular proteins or receptors of interest—particularly those whose expression is increased in cancerous tissue may be used. Such proteins or receptors of interest include, but are not limited to tumor antigens, such as CEA, which is expressed in colorectal carcinomas, Her2/neu, which is expressed in multiple cancers, BRCA 1 and BRCA 2, expressed in breast and ovarian cancers; and TRP-1 and -2, expressed in melanoma.

For example, human prostate-specific membrane antigen (PSMA) is upregulated in prostate cancer, including metastatic disease. PSMA is expressed by virtually all prostate cancers and its expression is further increased in poorly differentiated, metastatic and hormone refractory carcinomas. Accordingly, radiopharmaceuticals that comprise PSMA binding agents (e.g., compounds that a high affinity to PSMA) labelled with one or more radionuclide(s) can be used to obtain nuclear medicine images of a patient from which the presence and/or state of prostate cancer within a variety of regions (e.g., including, but not limited to skeletal regions) of the patient can be assessed. In certain embodiments, nuclear medicine images obtained using PSMA binding agents are used to identify the presence of cancerous tissue within the prostate, when the disease is in a localized state. In certain embodiments, nuclear medicine images obtained using radiopharmaceuticals comprising PSMA binding agents are used to identify the presence of cancerous tissue within a variety of regions that include not only the prostate, but also other organs and tissue regions such as lungs, lymph nodes, and bones, as is relevant when the disease is metastatic.

In particular, upon administration to a patient, radionuclide labelled PSMA binding agents selectively accumulate within cancerous tissue, based on their affinity to PSMA. In a similar manner to that described above with regard to ^{99m}Tc MDP, the selective concentration of radionuclide labelled PSMA binding agents at particular sites within the patient produces detectable hotspots in nuclear medicine images. As PSMA binding agents concentrate within a variety of cancerous tissues and regions of the body expressing PSMA, localized cancer within a prostate of the patient and/or metastatic cancer in various regions of the patient's body can be detected, and evaluated. Various metrics that are indicative of and/or quantify severity (e.g., likely malignancy) of individual lesions, overall disease burden and risk for a patient, and the like, can be computed based on automated analysis of intensity variations in nuclear medicine images obtained following administration of a PSMA binding agent radiopharmaceutical to a patient. These disease burden and/or risk metrics may be used to stage disease and make assessments regarding patient overall survival and other prognostic metrics indicative of disease state, progression, treatment efficacy,

A variety of radionuclide labelled PSMA binding agents may be used as radiopharmaceutical imaging agents for nuclear medicine imaging to detect and evaluate prostate cancer. In certain embodiments, the particular radionuclide labelled PSMA binding agent that is used depends on factors such as the particular imaging modality (e.g., PET; e.g., SPECT) and the particular regions (e.g., organs) of the patient to be imaged. For example, certain radionuclide labelled PSMA binding agents are suited for PET imaging, while others are suited for SPECT imaging. For example, certain radionuclide labelled PSMA binding agents facilitate imaging a prostate of the patient, and are used primarily when the disease is localized, while others facilitate imaging organs and regions throughout the patient's body, and are useful for evaluating metastatic prostate cancer.

Several exemplary PSMA binding agents and radionuclide labelled versions thereof are described in further detail in Section H herein, as well as in U.S. Pat. Nos. 8,778,305, 8,211,401, and 8,962,799, and in U.S. Patent Publication No. US 2021/0032206 A1, the content of each of which are incorporated herein by reference in their entireties.

### B. Image Segmentation in Nuclear Medicine Imaging

Nuclear medicine images are functional images. Functional images convey information relating to physiological activities within specific organs and/or tissue, such as metabolism, blood flow, regional chemical composition, and/or absorption. In certain embodiments, nuclear medicine images are acquired and/or analyzed in combination with anatomical images, such as computed tomography (CT) images. Anatomical images provide information regarding location and extent of anatomical structures such as internal organs, bones, soft-tissue, and blood vessels, within a subject. Examples of anatomical images include, without limitation, x-ray images, CT images, magnetic resonance images, and ultrasound images.

Accordingly, in certain embodiments, anatomical images can be analyzed together with nuclear medicine images in order to provide anatomical context for the functional information that they (nuclear medicine images) convey. For example, while nuclear medicine images, such as PET and SPECT convey a three-dimensional distribution of radiopharmaceutical within a subject, adding anatomical context from an anatomical imaging modality, such as CT imaging, allows one to determine the particular organs, soft-tissue regions, bones, etc. that radiopharmaceutical has accumulated in.

For example, a functional image may be aligned with an anatomical image so that locations within each image that correspond to a same physical location—and therefore correspond to each other—can be identified. For example, coordinates and/or pixels/voxels within a functional image and an anatomical image may be defined with respect to a common coordinate system, or a mapping (i.e., a functional relationship) between voxels within the anatomical image and voxels within the functional image established. In this manner, one or more voxels within an anatomical image and one or more voxels within a functional image that represent a same physical location or volume can be identified as corresponding to each other.

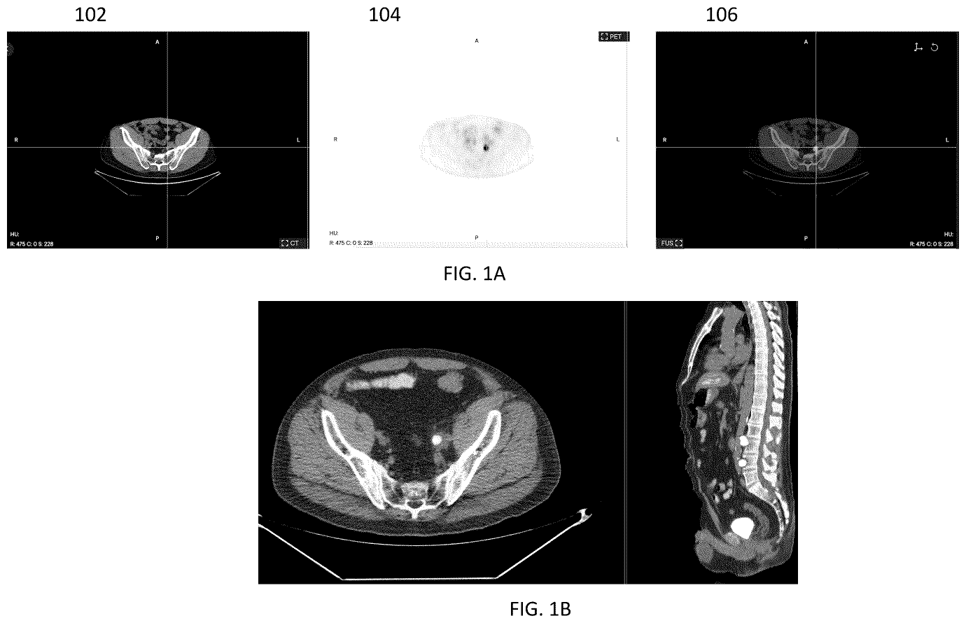

For example, FIG. 1 shows axial slices of a 3D CT image 102 and a 3D PET image 104, along with a fused image 106 in which the slice of the 3D CT image is displayed in grayscale and with the PET image is displayed as a semitransparent overlay. By virtue of the alignment between the CT and PET images, a location of a hotspot within the PET image, indicative of accumulated radiopharmaceutical and, accordingly a potential lesion, can be identified in the corresponding CT image, and viewed in anatomical context, for example, within a particular location in the pelvic region (e.g., within a prostate). FIG. 1B shows another PET/CT fusion, showing a transvers plane slice and a sagittal plane slice.

In certain embodiments, the aligned pair are a composite image, such as a PET/CT or SPECT/CT. In certain embodiments, an anatomical image (e.g., a 3D anatomical image, such as a CT image) and a functional image (e.g., a 3D functional image, such as a PET or SPECT image) are acquired using separate anatomical and functional imaging modalities, respectively. In certain embodiments, an anatomical image (e.g., a 3D anatomical image, such as a CT image) and a functional image (e.g., a 3D functional image, such as a PET or SPECT image) are acquired using a single multimodality imaging system. A functional image and an anatomical image may, for example, be acquired via two scans using a single multimodal imaging system—for example first performing a CT scan and then, second, performing a PET scan—during which a subject remains in a substantially fixed position.

In certain embodiments, 3D boundaries of particular tissue regions of interest can be accurately identified by analyzing 3D anatomical images. For example, automated segmentation of 3D anatomical images can be performed to segment 3D boundaries of regions such as particular organs, organ sub-regions and soft-tissue regions, as well as bone. In certain embodiments, organs such as a prostate, urinary bladder, liver, aorta (e.g., portions of an aorta, such as a thoracic aorta), a parotid gland, etc., are segmented. In certain embodiments, one or more particular bones are segmented. In certain embodiments, an overall skeleton is segmented.

In certain embodiments, automated segmentation of 3D anatomical images may be performed using one or more machine learning modules that are trained to receive a 3D anatomical image and/or a portion thereof, as input, and segment one or more particular regions of interest, producing a 3D segmentation map as output. For example as described in PCT publication WO/2020/144134, entitled “Systems and Methods for Platform Agnostic Whole Body Segmentation,” and published Jul. 16, 2020, the contents of which are incorporated herein by reference in their entirety, multiple machine learning modules implementing convolutional neural networks (CNNs) may be used to segment 3D anatomical images, such as CT images, of a whole body of a subject and thereby create a 3D segmentation map that identifies multiple target tissue regions across a subject's body.

In certain embodiments, for example to segment certain organs where functional images are believed to provide additional useful information that facilitate segmentation, a machine learning module may receive both an anatomical image and a functional image as input, for example as two different channels of input (e.g., analogous to multiple color channels in a color, RGB, image) and use these two inputs to determine an anatomical segmentation. This, multi-channel, approach is described in further detail in U.S. Patent Publication No. US 2021/0334974 A1, entitled “Systems and Methods for Deep-Learning-Based Segmentation of Composite Images,” and published Oct. 28, 2021, the contents of which is hereby incorporated by reference in its entirety.

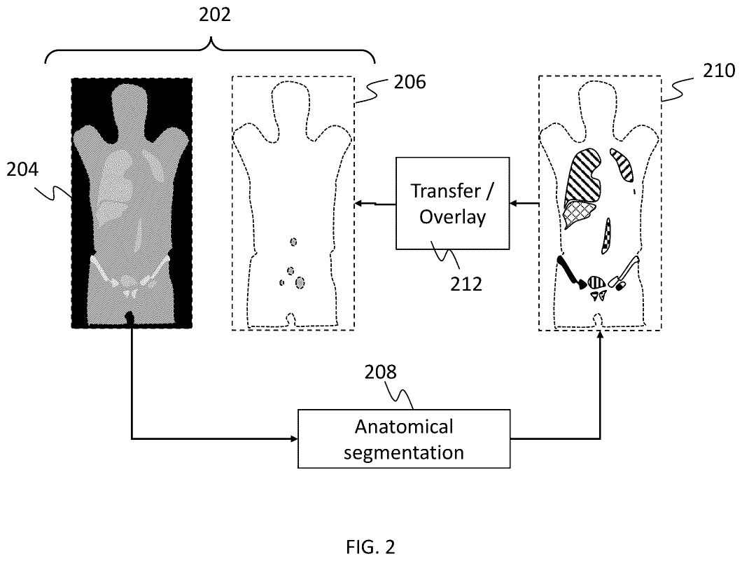

In certain embodiments, as illustrated FIG. 2, an anatomical image 204 (e.g., a 3D anatomical image, such as a CT image) and a functional image 206 (e.g., a 3D functional image, such as a PET or SPECT image) may be aligned with (e.g., co-registered to) each other, for example as in a composite image 202 such as a PET/CT image. Anatomical image 204 may be segmented 208 to create a segmentation map 210 (e.g., a 3D segmentation map) that distinguishably identifies one or more tissue regions and/or sub-regions of interest, such as one or more particular organs and/or bones. Segmentation map 210, having been created from anatomical image 204 is aligned with anatomical image 204, which, in turn, is aligned with functional image 206. Accordingly, boundaries of particular regions (e.g., segmentation masks), such as particular organs and/or bones, identified via segmentation map 210 can be transferred to and/or overlaid 212 upon functional image 206 to identify volumes within functional image 206 for purposes of classifying hotspots, and determining useful indices that serve as measures and/or predictions of cancer status, progression, and response to treatment. Segmentation maps and masks may also be displayed, for example as a graphical representation overlaid on a medical image to guide physicians and other medical practitioners.

### C. Lesion Detection and Characterization

In certain embodiments, approaches described herein include techniques for detecting and characterizing lesions within a subject via (e.g., automated) analysis of medical images, such as nuclear medicine images. As described herein, in certain embodiments, hotspots are localized (e.g., contiguous) regions of high intensity, relative to their surroundings, within images, such as 3D functional images and may be indicative of a potential cancerous lesion present within a subject.

A variety of approaches may be used for detecting, segmenting, and classifying hotspots. In certain embodiments, hotspots are detected and segmented using analytical methods, such as filtering techniques including, but not limited to, a difference of Gaussians (DoG) filter and a Laplacian of Gaussians (LoG) filter. In certain embodiments, hotspots are segmented using a machine learning module that receives, as input, a 3D functional image, such as a PET image, and generates, as output a hotspot segmentation map (a “hotspot map”) that differentiates boundaries of identified hotspots from background. In certain embodiments, each segmented hotspot within a hotspot map is individually identifiable (e.g., individually labelled). In certain embodiments, a machine learning module used for segmenting hotspots may take as input, in addition to a 3D functional image, one or both of a 3D anatomical image (e.g., a CT image) and a 3D anatomical segmentation map. The 3D anatomical segmentation map may be generated via automated segmentation (e.g., as described herein) of the 3D anatomical image.

In certain embodiments, segmented hotspots may be classified according to an anatomical region in which they are located. For example, in certain embodiments, locations of individual segmented hotspots within a hotspot map (representing and identifying segmented hotspots) may be compared with 3D boundaries of segmented tissue regions, such as various organs and bones, within a 3D anatomical segmentation map and labeled according to their location, e.g., based on proximity to and/or overlap with particular organs. In certain embodiments, a machine learning module may be used to classify hotspots. For example, in certain embodiments, a machine learning module may generate, as output, a hotspot map in which segmented hotspots are not only individually labeled and identifiable (e.g., distinguishable from each other), but are also labeled, for example, as corresponding to one of a bone, lymph, or prostate lesion. In certain embodiments, one or more machine learning modules may be combined with each other, as well as with analytical segmentation (e.g., thresholding) techniques to perform various tasks in parallel and in sequence to create a final labeled hotspot map.

Various approaches for performing detailed segmentation of 3D anatomical images and identification of hotspots representing lesions in 3D functional images, which may be used with various approaches described herein, are described in PCT publication WO/2020/144134, entitled “Systems and Methods for Platform Agnostic Whole Body Segmentation,” and published Jul. 16, 2020, U.S. Patent Publication No. US 2021/0334974 A1, entitled “Systems and Methods for Deep-Learning-Based Segmentation of Composite Images,” and published Oct. 28, 2021, and PCT publication WO/2022/008374, entitled “Systems and Methods for Artificial Intelligence-Based Image Analysis for Detection and Characterization of Lesions,” and published Jan. 13, 2022, the contents of each of which is incorporated herein in its entirety.

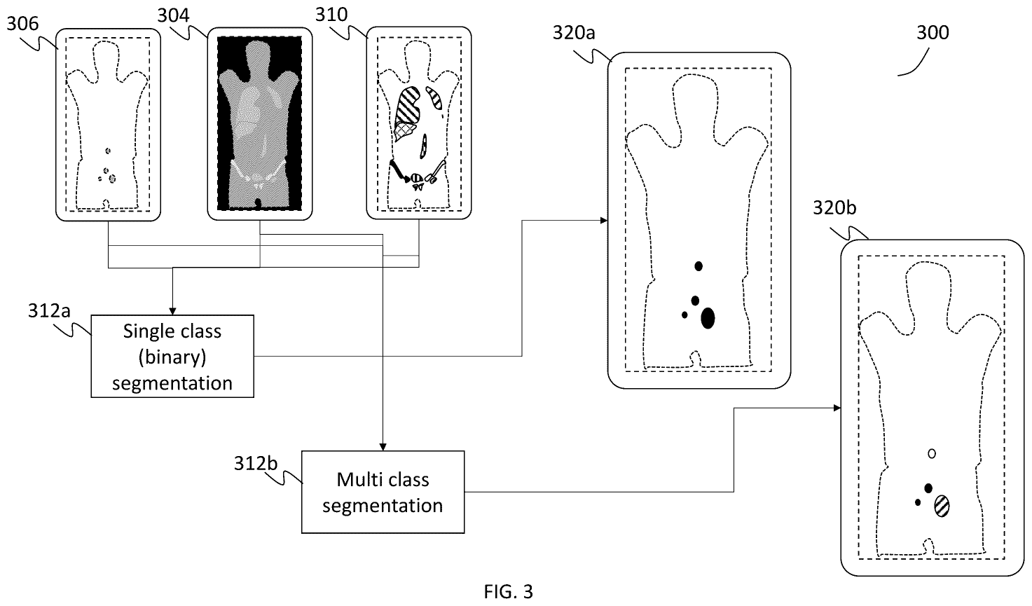

FIG. 3 shows an example process 300 for segmenting and classifying hotspots, based on an example approach described in further detail in PCT publication WO/2022/008374, entitled “Systems and Methods for Artificial Intelligence-Based Image Analysis for Detection and Characterization of Lesions,” and published Jan. 13, 2022. The approach illustrated in FIG. 3 uses two machine learning modules, each of which receives, as input, 3D functional image 306, 3D anatomical image 304, and 3D anatomical segmentation map 310. Machine learning module 312a is a binary classifier that generates a single-class hotspot map 320a, by labeling voxels as hotspot or background (not a hotspot). Machine learning module 312b performs multi-class segmentation, and generates multi-class hotspot map 320b, in which hotspots are both segmented and labeled as one of three classes—prostate, lymph, or bone. Among other things, classifying hotspots in this manner—via a machine learning module 312b (e.g., as opposed to directly comparing hotspot locations with segmented boundaries from segmentation map 310)—obviates a need to segment certain regions. For example, in certain embodiments, machine learning module 312b may classify hotspots as belonging to prostate, lymph, or bone, without a prostate region having be identified and segmented from 3D anatomical image 304 (e.g., in certain embodiments, 3D anatomical segmentation map 310 does not comprise a prostate region). In certain embodiments, hotspot maps 320a and 320b are merged, for example by transferring labels from multi-class hotspot map 320b to the hotspot segmentations identified in single-class hotspot map 320a (e.g., based on overlap). Without wishing to be bound to any particular theory, it is believed that this approach combines improved segmentation and detection of hotspots from single class machine learning module 312a with classification results from multi-class machine learning module 312b. In certain embodiments, hotspot regions identified via this final, merged, hotspot map are further refined, using an analytical technique such as an adaptive thresholding technique described in PCT publication WO/2022/008374, entitled “Systems and Methods for Artificial Intelligence-Based Image Analysis for Detection and Characterization of Lesions,” and published Jan. 13, 2022.

In certain embodiments, once detected and segmented, hotspots may be identified and assigned labels according to a particular anatomical (e.g., tissue) region in which they are located and/or a particular lesion sub-type that they are likely to represent. For example, in certain embodiments, hotspots may be assigned an anatomical location that identifies them as representing locations with a one of a set of tissue regions, such as the listed in Table 1, below. In certain embodiments, a list of tissue regions may include those in Table 1 as well as a gluteus maximus (e.g., left and right) and a gallbladder. In certain embodiments, hotspots are assigned to and/or labeled as belonging to a particular tissue region based on a machine learning classification and/or via comparison of their 3D hotspot volume's location and/or overlap with various tissue volumes identified via masks in an anatomical segmentation map. In certain embodiments, a prostate is not segmented. For example, as described above, in certain embodiments, machine learning module 312b may classify hotspots as belonging to prostate, lymph, or bone, without a prostate region having be identified and segmented from 3D anatomical image 304.

TABLE 1
Certain Tissue Regions (*Prostate may, optionally, be segmented if
present - may be absent if patient has, e.g., undergone radical
prostatectomy, or may not segmented in any case, in certain embodiments)
Organs/Bones

Right and Right Lung
Left and Right Femur
Left and Right Hip Bone
Urinary bladder
Sacrum and coccyx
Liver
Spleen
Left and Right Kidney
Left Side and Right Side Ribs 1-12
Left and Right Scapula
Left and Right Clavicle
Cervical vertebrae
Thoracic vertebrae 1-12
Lumbar vertebrae 1-5
Sternum
Aorta, thoracic part
Aorta, abdominal part
Prostate*

In certain embodiments, additionally or alternatively, hotspots may be classified as belonging to one or more lesion sub-types. In certain embodiments, lesion sub-type classifications may be made by comparing hotspot locations with classes of anatomical regions. For example, in certain embodiments a miTNM classification scheme may be used, where hotspots are labeled as belonging to one of three classes—miT, miN, or miM—based on whether they represent lesions located within a prostate (miT), pelvic lymph node (miN), or distant metastases (miM). In certain embodiments, a five class version of the miTNM scheme may be used, with distant metastases further divided into three sub classes—miMb for bone metastases, miMa for lymph metastases, and miMc for other soft tissue metastases.

For example, in certain embodiments, hotspots located within a prostate are labeled as belonging to class “T” or “miT”, e.g., representing local tumor. In certain embodiments, hotspots located outside a prostate, but within a pelvic region are labeled as class “N” or “miN”. In certain embodiments, for example as described in U.S. application Ser. No. 17/959,357, filed Oct. 4, 2022, entitled “Systems and Methods for Automated Identification and Classification of Lesions in Local Lymph and Distant Metastases,” published as U.S. 2023/0115732 A1 on Apr. 13, 2023, the content of which is incorporated herein by reference in its entirety, a pelvic atlas may be registered to identify boundaries of a pelvic region and/or various sub-regions therein, for purposes of identifying pelvic lymph node lesions. A pelvic atlas may, for example, include boundaries of a pelvic region and/or a planar reference (e.g., a plane passing through an aorta-bifurcation) which hotspot locations can be compared to (e.g., such that hotspots located outside the pelvic region and/or above the planar reference passing through an aorta bifurcation are labeled as “M” or “miM”—e.g., distant metastases). In certain embodiments, distant metastases may be classified as lymph (miMa), bone (miMb), or visceral (miMc) based on a comparison of hotspot locations with an anatomical segmentation map. For example, hotspots located within one or more bones (e.g., and outside a pelvic region) may be labeled as distant metastases, hotspots located within one or more segmented organs or a subset of organs (e.g., brain, lung, liver, spleen, kidneys) may be labeled as visceral (miMc) distant metastases, and remaining hotspots located outside a pelvic region labeled as distant lymph metastases (miMa).

Additionally or alternatively, in certain embodiments, hotspots may be assigned an miTNM class based on a determination that they are located within a particular anatomical region, for example based on a table such as Table 2, where each column corresponds to a particular miTNM label (first row indicating the particular miTNM class) and includes, in rows two and below, particular anatomical regions associated with each miTNM class. In certain embodiments, a hotspot can be assigned as being located within a particular tissue region listed in Table 2 based on a comparison of the hotspot's location with an anatomical segmentation map, allowing for an automated miTNM class assignment.

TABLE 2
An Example List of Tissue Regions Corresponding to Five
Classes in a Lesion Anatomical Labeling Approach

| | | Pelvic lymph | | |
|---|---|---|---|---|
| Bone | Lymph nodes | nodes | Prostate | Visceral |
| Mb | Ma | N | T | Mc |
| Skull | Cervical | Template right | Prostate | Brain |
| Thorax | Supraclavicular | Template left | | Neck |
| Vertebrae | Axillary | Presacral | | Lung |
| lumbar | | | | |
| Vertebrae | Mediastinal | Other, pelvic | | Esophageal |
| thoracic | | | | |
| Pelvis | Hilar | | | Liver |
| Extremities | Mesenteric | | | Gallbladder |
| | Elbow | | | Spleen |
| | Popliteal | | | Pancreas |
| | Peri-/para-aortic | | | Adrenal |
| | Other, non-pelvic | | | Kidney |
| | | | | Bladder |
| | | | | Skin |
| | | | | Muscle |
| | | | | Other |

In certain embodiments, hotspots may be further classified in terms of their anatomical location and/or lesion sub-type. For example, in certain embodiments, hotspots identified as located in pelvic lymph (miN) may be identified as belonging to a particular pelvic lymph node sub-region, such as one of a left/right internal iliac, a left or right external iliac, a left or right common iliac, a left or right obturator, a presacral region, or other pelvic region. In certain embodiments, distant lymph node metastases (miMa) may be classified as retroperitoneal (RP), supradiaphragmatic (SD), or other extrapelvic (OE). Approaches for regional (miN) and distant (miMa) lymph metastases classifications may include registration of pelvic atlas images and/or identification of various whole body landmarks, which are described in further detail in U.S. application Ser. No. 17/959,357, filed Oct. 4, 2022, entitled “Systems and Methods for Automated Identification and Classification of Lesions in Local Lymph and Distant Metastases,” published as U.S. 2023/0115732 A1 on Apr. 13, 2023, the content of which is incorporated herein by reference in its entirety.

### D. Individual Hotspot Quantification Metrics

In certain embodiments, detected—e.g., identified and segmented—hotspots may be characterized via various individual hotspot quantification metrics. In particular, for a particular individual hotspot, individual hotspot quantification metrics can be used to quantify a measure of size (e.g., 3D volume) and/or intensity of the particular hotspot in a manner that is indicative of a size and/or level of radiopharmaceutical uptake within the (e.g., potential) underlying physical lesion that the particular hotspot represents. Accordingly, individual hotspot quantification metrics may convey, for example to a physician or radiologist, a likelihood that a hotspot appearing in an image represents a true underlying physical lesion and/or convey a likelihood or level of malignancy thereof (e.g., allowing to differentiate between benign and malignant lesions).

In certain embodiments, image segmentation, lesion detection, and characterization techniques as described herein are used to determine, for each of one or more medical images, a corresponding set of hotspots. As described herein, image segmentation techniques may be used to determine, for each hotspot detected in a particular image, a particular 3D volume—a 3D hotspot volume—representing and/or indicative of a volume (e.g., 3D location and extent) of a potential underlying physical lesion within the subject. Each 3D hotspot volume, in turn, comprises a set of image voxels, each having a particular intensity value.

Once determined, a set of 3D hotspot volumes may be used to compute one or more hotspot quantification metrics for each individual hotspot. Individual hotspot quantification metrics may be computed according to various methods and formulae described herein, for example below. In the description below, the variable L is used to refer to a set of hotspots detected with a particular image, with L={1, 2, . . . , l, . . . , N_{L}} representing a set of N_{L} (i.e., N_{L} being the number of hotspots) hotspots detected within an image and the variable l indexing the l^{th} hotspot. As described herein, each hotspot corresponds to a particular 3D hotspot volume within an image, with Rₗ denoting the 3D hotspot volume of the l^{th} hotspot.

Hotspot quantification metrics may be presented to a user via a graphical user interface (GUI) and/or a (e.g., automatically or semi-automatically) generated report. As described in further detail herein, individual hotspot quantification metrics may include hotspot intensity metrics and hotspot volume metrics (e.g., lesion volume) that quantify an intensity and size, respectively of a particular hotspot and/or underlying lesion it represents. Hotspot intensity and size may, in turn, be indicative of a level of radiopharmaceutical uptake within, and size of, respectively, an underlying physical lesion within the subject.

D.i. Hotspot Intensity Metrics

In certain embodiments, a hotspot quantification metric is or comprises an individual hotspot intensity metric that quantifies an intensity of an individual 3D hotspot volume. Hotspot intensity metrics may be computed based on individual voxel intensities within identified hotspot volumes. For example, for a particular hotspot, a value of a hotspot intensity metric may be computed as a function of at least a portion (e.g., a particular subset, e.g., all) of that hotspot's voxel intensities. Hotspot intensity metrics may include, without limitation, metrics such as a maximum hotspot intensity, a mean hotspot intensity, and peak hotspot intensity, and the like. As with voxel intensities in nuclear medicine images, in certain embodiments hotspot intensity metrics may represent (e.g., be in units of) SUV values.

In certain embodiments, a value of a particular hotspot intensity metric are computed, for a subject hotspot, based on (e.g., as a function of) that subject hotspot's voxel intensities alone, e.g., and not based on intensities of other image voxels outside the subject hotspot's 3D volume.

For example, a hotspot intensity metric may be a maximum hotspot intensity (e.g., SUV), or “SUV-max,” computed as a maximum voxel intensity (e.g., SUV or uptake) within a 3D hotspot volume. In certain embodiments, a maximum hotspot intensity may be computed according to equations (1a), (1b), or (1c), below

$${Q}_{max} ( l ) = {max}_{i ∈ {R}_{l}} ( {q}_{i} ) \qquad ( 1 a )$$

$${SUV}_{max} ( l ) = {max}_{i ∈ {R}_{l}} ( {SUV}_{i} ) \qquad ( 1 b )$$

$$SUV = max ( UptakeInVoxel ∈ lesion volume ) \qquad ( 1 c )$$

where, in equations (1a) and (1b) l represents a particular (e.g., l^{th}) hotspot, as described above, qᵢ is the intensity of voxel i and i ∈Rₗ is the set of voxels within the particular 3D hotspot volume, Rₗ. In equation (1b), SUVᵢ indicates a particular unit—standard uptake value (SUV)—of voxel intensity, as described herein.

In certain embodiments, a hotspot intensity metric may be a mean hotspot intensity (e.g., SUV), or “SUV-mean,” and may be computed as a mean over all voxel intensities (e.g., SUV or uptake) within a 3D hotspot volume. In certain embodiments, a mean hotspot intensity may be computed according to equations (2a), (2b), or (2c) below.

$${Q}_{mean} ( l ) = {mean}_{i ϵ {R}_{l}} ( {q}_{i} ) = \frac{1}{{n}_{l}} {\sum }_{i ϵ {R}_{l}} {q}_{i} \qquad ( 2 a )$$

$${SUV}_{mean} ( l ) = {mean}_{i ϵ {R}_{l}} ( {SUV}_{i} ) = \frac{1}{{n}_{l}} {\sum }_{i ϵ {R}_{l}} {SUV}_{i} \qquad ( 2 b )$$

$${SUV}_{mean} = {\sum }_{i ∈ lesion volume} \frac{UptakeInVoxel}{{n}_{l}} \qquad ( 2 c )$$

where nₗ is the number of individual voxels within a particular 3D hotspot volume.

In certain embodiments, a hotspot intensity metric may be a peak hotspot intensity (e.g., SUV), or “SUV-peak,” and may be computed as a mean over intensities of the voxels (e.g., SUV or uptake) whose midpoints are located within a (e.g., pre-defined) particular distance (e.g., within 5 mm) of the midpoint of the hotspot voxel where the maximum intensity (e.g., SUV-max) is located within a hotspot, and, accordingly, may be computed according to equations (3a)-(3c) below.

$${Q}_{peak} ( l ) = \frac{1}{{n}_{l}} {{\sum }_{i :}}_{dist ( {i}_{max} , i ) \leq d} {q}_{i} \qquad ( 3 a )$$

$${SUV}_{peak} ( l ) = \frac{1}{{n}_{l}} {{\sum }_{i :}}_{dist ( {i}_{max} , i ) \leq d} {SUV}_{i} \qquad ( 3 b )$$

$${SUV}_{peak} = \frac{1}{{n}_{l}} {{{\sum }_{i :}}_{dist ( {SUV}_{max} point , i )}}_{\leq 5 mm} {UptakeInVoxel}_{i} \qquad ( 3 c )$$

where i: dist(iₘₐₓ,i)≤d is the set of (hotspot) voxels having a mid-point within a distance, d, from voxel iₘₐₓ, which is the maximum intensity voxel within the hotspot (e.g., Qₘₐₓ(l)=qᵢ₋ₘₐₓ.
D.ii. Lesion Index Metrics

In certain embodiments, a hotspot intensity metric is individual lesion index value that maps an intensity of voxels within a particular 3D hotspot volume to a value on a standardized scale. Such lesion index values are described in further detail in PCT/EP2020/050132, filed Jan. 6, 2020, and PCT/EP2021/068337, filed Jul. 2, 2021, the content of each of which is hereby incorporated by reference in its entirety. Calculation of lesion index values may include calculation of reference intensity values within particular reference tissue regions, such as an aorta portion (also referred to as blood pool) and/or a liver.

For example, in one particular implementation, a first, blood-pool, reference intensity value is determined based on a measure of intensity (e.g., a mean SUV) within an aorta region and a second, liver, reference intensity value is determined based on a measure of intensity (e.g., a mean SUV) within a liver region. As described in further detail, for example in PCT/EP2021/068337, filed Jul. 2, 2021, the content of which is incorporated herein by reference in its entirety, calculation of reference intensities may include approaches such as identifying reference volumes (e.g., an aorta or portion thereof, e.g., a liver volume) within a functional image, such as a PET or SPECT image, eroding and/or dilating certain reference volumes, e.g., to avoid include voxels on the edge of a reference volume, and selecting subsets of reference voxel intensities, based on modeling approaches, e.g., to account for anomalous tissue features, such as cysts and lesions, within a liver. In certain embodiments, a third reference intensity value may be determined, either as a multiple (e.g., twice) of a liver reference intensity value, or based on an intensity of another reference tissue region, such as a parotid gland.

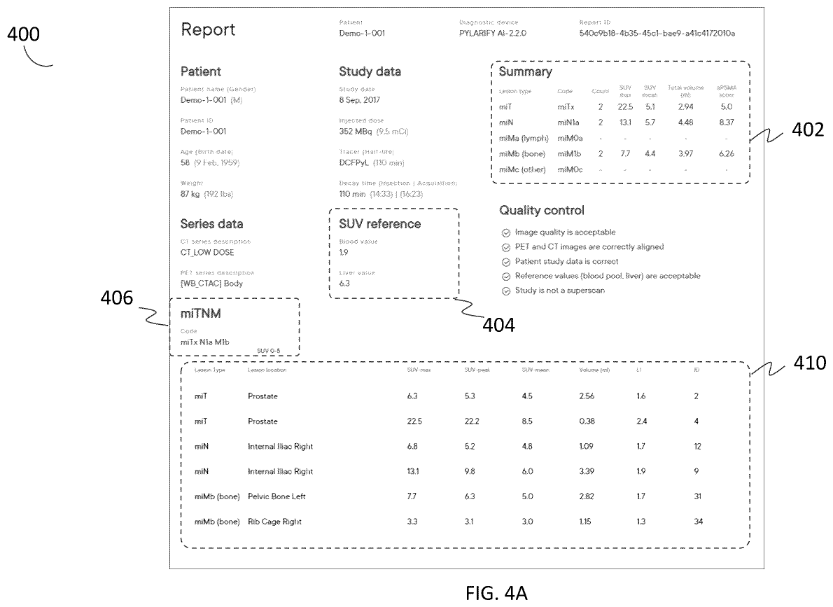

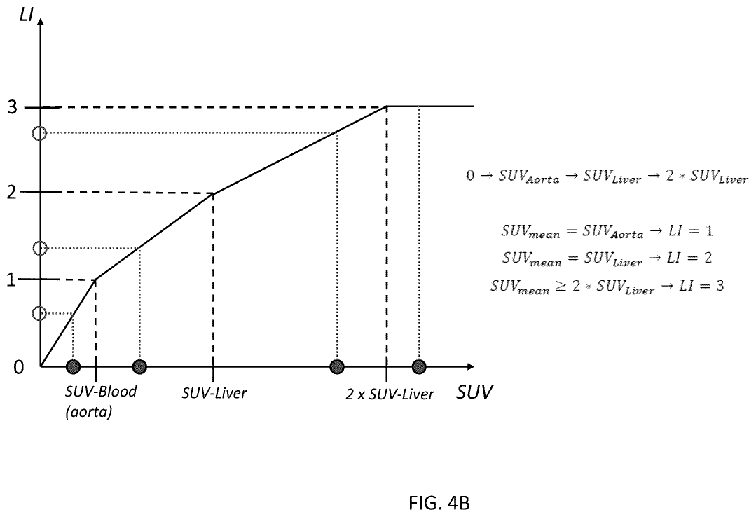

In certain embodiments, hotspot intensities may be compared with one or more reference intensity values to determine a lesion index as a value on a standardized scale, which facilitates comparison across different images. For example, FIG. 4B illustrates an approach for assigning hotspots a lesion index value ranging from 0 to 3. In the approach shown in FIG. 4B, a blood-pool (aorta) intensity value is assigned a lesion index of 1, a liver intensity value is assigned a lesion 2, and a value of twice the liver intensity is assigned a lesion index of 3. A lesion index for a particular hotspot can be determined by first computing a value of an initial hotspot intensity metric for the particular hotspot, such as a mean hotspot intensity (e.g., Qₘₑₐₙ(l) or SUVₘₑₐₙ) and comparing the value of the initial hotspot intensity metric with the reference intensity values. For example, the value of the initial hotspot intensity metric may fall within one of four ranges—[0, SUV_{blood}], (SUV_{blood}, SUVₗᵢᵥₑᵣ], (SUVₗᵢᵥₑᵣ, 2×SUVₗᵢᵥₑᵣ], and greater than 2×SUVₗᵢᵥₑᵣ (e.g., (2×SUVₗᵢᵥₑᵣ, ∞)). A lesion index value can then be computed for the particular hotspot based on (i) the value of the initial hotspot intensity metric and (ii) a linear interpolation according to the particular range in which the value of the initial hotspot intensity metric falls, as illustrated in FIG. 4B, where the filled and open dots on the horizontal (SUV) and vertical (LI) axes illustrate example values of initial hotspot intensity metrics and resultant lesion index values, respectively. In certain embodiments, if SUV references for either liver or aorta cannot be calculated, or if the aorta value is higher than the liver value, the Lesion Index will not be calculated and will be displayed as ‘−’.

A lesion index value according to the mapping scheme described above and illustrated in FIG. 4B may, for example, be computed as shown in equation (4), below.

$${Q}_{LI} ( l ) = \{ \begin{matrix}{f}_{1} ( {SUV}_{mean} ( l ) ) , & {SUV}_{mean} ( l ) \leq {SUV}_{aorta} \\ {f}_{2} ( {SUV}_{mean} ( l ) ) , & {SUV}_{aorta} \leq {SUV}_{mean} ( l ) \leq {SUV}_{liver} \\ {f}_{3} ( {SUV}_{mean} ( l ) ) , & {SUV}_{liver} \leq {SUV}_{mean} ( l ) \leq 2 \times {SUV}_{liver} \\ 3 , & 2 \times {SUV}_{liver} \leq {SUV}_{mean} ( l )\end{matrix} \qquad ( 4 )$$

where ƒ₁ ƒ₂ and ƒ₃ are linear interpolations between the respective spans in equation (4).
D.iii. Hotspot Lesion Volume

In certain embodiments, a hotspot quantification metric may be a volume metric, such as a lesion volume, Qᵥₒₗ, which provides a measure of size (e.g., volume) of an underlying physical lesion that a hotspot represents. A lesion volume may, in certain embodiments, computed as shown in equations (5a) and (5b), below.

$${Q}_{vol} ( l ) = {\sum }_{i ∈ {R}_{l}} {v}_{i} \qquad ( 5 a )$$

$${Q}_{vol} ( l ) = v \times {n}_{l} \qquad ( 5 b )$$

where in equation (5a), vᵢ is a volume of an i^{th} voxel, and equation (5b) assumes a uniform voxel volume, v, and as before nₗ is a number of voxels in a particular hotspot volume, l. In certain embodiments, a voxel volume is computed as v=δx×δy×δz, where δx, δy, and δz are grid spacing (e.g., in millimeters, mm) in x, y, and z. In certain embodiments, a lesion volume has units of milliliters (ml).

### E. Aggregating Hotspot Metrics

In certain embodiments, systems and methods described herein compute patient index values that quantify disease burden and/or risk for a particular subject. Values of various patient indices may be computed using, for example as a function of, values of individual hotspot quantification measures. In particular, in certain embodiments, a particular patient index value aggregates values of multiple individual hotspot quantification metrics computed for an entire set of hotspots detected for the patient and/or for a particular subset of hotspots, for example associated with particular tissue regions and/or lesion sub-types. In certain embodiments, a particular patient index is associated with one or more specific individual hotspot quantification measures and is computed using the (e.g., multiple) values of the specific individual hotspot quantification metrics computed for each of at least a portion of the individual 3D hotspot volumes in the set.

E.i. Overall Patient Indices

For example, in certain embodiments, a particular patient index may be an overall patient index that aggregates values of one or more specific individual hotspot quantification measures computed across substantially an entire set of 3D hotspot volumes detected for a patient at a particular time point, to, for example, provide an overall measure of total disease burden for the subject at the particular time point.

In certain embodiments, a particular patient index may be associated with a single specific individual hotspot quantification measure and may be computed as a function of substantially all values of that specific individual hotspot quantification measure for the set of 3D hotspot volumes. Such patient indices may be viewed has having a functional form,

$${P}_{p , m} = {f}_{( p )} ( {Q}_{( m ) , L} ) \qquad ( 6 )$$

where Q₍ₘ₎, denotes a particular individual hotspot quantification metric, such as Qₘₐₓ, Qₘₑₐₙ, Qₚₑₐₖ, Qᵥₒₗ, Q_{LI}, as described above, and Q_{(m),L} is the set of values of the specific individual hotspot quantification metric computed for each hotspot, l, in the set of hotspots L. That is, Q_{(m),L} is the set {Q₍ₘ₎(l=1), Q₍ₘ₎(1=2), . . . , Q₍ₘ₎(1−N_{L})}.

The function, ƒ₍ₚ₎, may be a variety of functions, which suitably aggregates (combines) the overall set of values of the particular specific individual hotspot quantification metric, Q₍ₘ₎. For example, the function ƒ₍ₚ₎ may be a sum, a mean, a median, a mode, a max, etc. Different particular functions may be used for ƒ₍ₚ₎, depending on the particular hotspot quantification metric, Q₍ₘ₎ that is being aggregated. Accordingly, various individual hotspot quantification measures (e.g., a mean intensity, a median intensity, a mode of intensities, a peak intensity, an individual lesion index, a volume) may be combined in a variety of manners, for example by taking an overall sum, mean, median, mode, etc., over substantially all values computed for the 3D hotspot volumes of the set.

For example, in certain embodiments, an overall patient index may be an overall intensity maximum, which is computed as a maximum over all individual hotspot maximum intensity values, as shown in equations (7a) or (7b), below

$${P}_{max} = max ( {Q}_{max , L} ) = {max}_{l ∈ L} {Q}_{max} ( l ) \qquad ( 7 a )$$

$${P}_{max} = max ( {SUV}_{max , L} ) = {max}_{l ∈ L} {SUV}_{max} ( l ) \qquad ( 7 b )$$

where Qₘₐₓ(l) may be computed according to equation (1a), above, in general, or according to equations (1b) or (1c) where image intensities represent SUV values, for example, as reflected in equation (7b).

In certain embodiments a particular patient index value may be computed as a combination of substantially all individual hotspot mean intensity values, for example as a sum of the mean intensity values, e.g., as shown in equations (8a) and (8b), below.

$${P}_{sum} = {\sum }_{l ∈ L} {Q}_{mean} ( l ) \qquad ( 8 a )$$

$${P}_{sum} = {\sum }_{l ∈ L} {SUV}_{mean} ( l ) \qquad ( 8 b )$$

In certain embodiments, an overall patient index is a total lesion volume, computed, for example, as a sum over all individual hotspot volumes, thereby providing a measure of total lesion volume. A total lesion volume may, for example, be computed as shown in equation (9a) and/or (9b), below,

$${P}_{Vol} = {\sum }_{l ∈ L} {Q}_{vol} ( l ) = {\sum }_{l ∈ L} {\sum }_{i ∈ {R}_{l}} {v}_{i} \qquad ( 9 a )$$

$${P}_{Vol} = {\sum }_{l ∈ L} {Q}_{vol} ( l ) = v {\sum }_{l ∈ L} {n}_{l} \qquad ( 9 b )$$

where (9b) assumes a uniform voxel size—i.e., each voxel has a same volume, vᵢ=v.

In certain embodiments, an overall patient index may be computed (e.g., directly) as a function of intensities, volumes, and/or number of voxels within the entire set of hotspots (e.g., as a function of all hotspot voxels within a union of all 3D hotspot volumes; e.g., not necessarily a function of individual hotspot quantification metrics). For example, in certain embodiments a patient index may be an overall mean value, and may be computed, for example, as shown in equations (10a) and (10b), below (i.e., by summing up intensities of all individual hotspot voxels for an entire set of hotspots, L, and dividing by a total number of hotspot voxels (for the entire set, L)).

$${P}_{mean} = \frac{{\sum }_{l ∈ L} {\sum }_{i ∈ {R}_{l}} {q}_{i}}{{\sum }_{l ∈ L} {n}_{l}} \qquad ( 10 a )$$

$${P}_{mean} = \frac{{\sum }_{l ∈ L} {\sum }_{i ∈ {R}_{l}} {SUV}_{i}}{{\sum }_{l ∈ L} {n}_{l}} \qquad ( 10 b )$$

In certain embodiments, a particular patient index may be computed using two or more specific individual hotspot quantification measures, e.g.,

$${P}_{p , m} = {f}_{( p )} ( {Q}_{( m 1 ) , L} , {Q}_{( m 2 ) , L} … ) \qquad ( 11 )$$

For example, an intensity-weighted measure of volume may be computed using both a measure of hotspot intensity and a measure of hotspot volume. For example, an intensity-weighted total volume may be computed at a patient level by computing, for each individual hotspot, a product of a lesion index computed for the individual hotspot and a volume of the hotspot. A sum over substantially all intensity-weighted volumes may then be computed to determine a total score according to, for example, the equation below, in which Q_{LI}(l) and Qᵥₒₗ(l) are the values of the individual lesion index and volume, respectively, for the i^{th} 3D hotspot volume.

$${P}_{ILV} = {\sum }_{l ∈ L} {Q}_{LI} ( l ) \times {Q}_{vol} ( l ) \qquad ( 12 )$$

Other measures of intensity, for example as described above, may be used to weight a hotspot volume or compute version other metrics. In certain embodiments, additionally or alternatively, a patient index may be determined by multiplying a total lesion volume (e.g., as computed in equations (9a) or (9b)) by total SUV mean (e.g., as computed in equations (10a) or (10b)) to provide an assessment that also combines intensity with volume.

In certain embodiments, a patient index is or comprises a total lesion count, computed a total number of substantially all hotspots detected (e.g., N_{L}).

E.ii. Region and Lesion Sub-Type Stratified Patient Indices

In certain embodiments, additionally or alternatively, multiple values of a particular patient index may be computed, each value associated with and computed for a particular subset of the 3D hotspot volumes (e.g., as opposed to the set L of substantially all hotspots).

In particular, in certain embodiments, 3D hotspot volumes within the set may be arranged in/assigned to one or more subsets according to, for example, particular tissue regions in which they are located, or a sub-type based on a classification scheme, such as the miTNM classification. Approaches for grouping hotspots according to tissue regions and/or according to an anatomical classification such as miTNM are described in further detail in in PCT/EP2020/050132, filed Jan. 6, 2020, and PCT/EP2021/068337, filed Jul. 2, 2021, the content of each of which is hereby incorporated by reference in its entirety.

In this manner, values of patient indices as described herein may be computed for one or more particular tissue regions, such as a skeletal region, a prostate, or a lymph region. In certain embodiments, lymph regions may be further stratified in a finely grained fashion, for example using approaches as described PCT/EP22/77505, filed Oct. 4, 2022 (published as WO2023/057411 on Apr. 13, 2023), the content of which is hereby incorporated by reference in its entirety. Additionally or alternatively, in certain embodiments, each 3D hotspot volume may be assigned a particular miTNM sub-type and grouped into subsets according to the miTNM classification, and values of various patient indices may be computed for each miTNM classification.

For example, where hotspots are assigned a particular lesion sub-type according to the miTNM staging system, miTNM class-specific versions of the overall patient indices described above. For example, in certain embodiments, hotspots may be identified (e.g., automatically, based on their location) as local tumor (T), intrapelvic nodes (N), or distant metastases (M), and assigned a label such as miT, miN, and miM, respectively, to identify three subsets. In certain embodiments, distant metastases may be further subdivided accordingly to whether the lesion appears (e.g., as determined by hotspot location) in a distant lymph node region (a), a bone (b), or other site, such as another organ (c). Hotspots may thus be assigned one of five lesion (e.g., miTNM) classes (e.g., miT, miN, miMa, miMb, miMc). Accordingly, each hotspot may be assigned to a particular subset, S, such that, for example, values of a patient index P(S) may be computed for each subset, S, of hotspots within an image. For example, equations (13a-d), below, can be used to calculate patient index values for particular subsets of hotspots.

$${P}_{max} ( S ) = max ( {Q}_{max , S} ) = {max}_{l ∈ S} {Q}_{max} ( l ) \qquad ( 13 a )$$

$${P}_{mean} ( S ) = \frac{{\sum }_{l ∈ S} {\sum }_{i ∈ {R}_{l}} {q}_{i}}{{\sum }_{l ∈ S} {n}_{l}} \qquad ( 13 b )$$

$${P}_{Vol} ( S ) = {\sum }_{l ∈ S} {Q}_{vol} ( l ) = v {\sum }_{l ∈ S} {n}_{l} \qquad ( 13 c )$$

$${P}_{ILV} ( S ) = {\sum }_{l ∈ S} {Q}_{LI} ( l ) \times {Q}_{vol} ( l ) \qquad ( 13 d )$$

where S denotes a particular subset of hotspots, such as local tumor (e.g., miT), intrapelvic nodes (e.g., labeled miN), distant metastases (e.g., labeled miM) or a particular type of distant metastases, such as a distant lymph node (e.g., labeled miMa), a bone (e.g., labeled miMb), or other site (e.g., labeled miMc). In each of equations (13a)-(13d), l ∈S denotes the hotspots within subset S. Equation (13a) is analogous to equation (7a), with Q_{max,S} denoting the maximum hotspot intensity for hotspots within the subset S, and where Qₘₐₓ(l) may be computed according to equation (1a), above, in general, or according to equations (1b) or (1c) where image intensities represent SUV values. Equation (13b) is analogous to equation (10a), with qᵢ denoting the intensity (which may be in SUV units) of the i^{th} voxel and the combined hotspot volume over which the mean is taken is the union of all hotspot volumes within subset S. Equation (13c) is analogous to equation (9b), and gives an overall lesion volume for a particular subset, S. Equation (13d) is analogous to equation (12), and provides an overall intensity weighted lesion volume over a particular subset, S.

In certain embodiments, a lesion count may be computed a number of substantially all detected hotspots within a particular subset, S (e.g., Ns).

E.iii. Scaled Patient Index Values

In certain embodiments, various patient index values may be scaled, for example accordingly to physical characteristics of a subject (e.g., weight, height, BMI, etc.) and/or volumes of tissue regions (e.g., a volume of a total skeletal region, a prostate volume, a total lymph volume, etc.) determined by analyzing (e.g., 3D anatomical images) images of the subject.

E.iv. Reporting Patient Index Values

Turning to FIG. 4A, patient index values computed as described herein may be displayed (e.g., in a chart, graph, table, etc.) in a report (e.g., an automatically generated report), such as an electronic document or a portion of a graphical user interface, for example for review and validation/sign-off by a user.

Among other things, as shown in FIG. 4A, a generated report 400 as described herein may include a summary of patient index values 402 that quantify disease burden in the patient, for example grouping hotspot subsets according to a lesion type (e.g., an miTNM classification) and displaying, for each lesion type, one or more computed patient index values for that sub-type. For example, summary portion 402 of report 400 displays patient index values for five subsets of hotspots, labeled miT, miN, miMa (lymph), miMb (bone), and miMc (other), based on the miTNM staging system. For each lesion sub-type, summary table 402 displays a number of detected hotspots belonging to that sub-type (e.g., within the particular subset), a maximum SUV (SUVₘₐₓ), a mean SUV (SUVmean), a total volume, and a quantity referred to as “aPSMA score”. For each lesion sub-type, S, values for SUVmax, SUVmean, Total volume, and aPSMA score may be computed as described above, for example, according to equations (13a), (13b), (13c), and (13d), respectively. In FIG. 4A, the term “aPSMA score” is used to reflect use of a PSMA binding agent, such as [18F]DCFPyL for imaging.

Summary table 402 in FIG. 4A also includes, for each lesion type, an alpha numeric code (e.g., miTx, miN1a, miM0a, miM1b, miM0c, displayed from top to bottom) that characterizes a severity, number, and location of lesions in the various regions, in accordance with the whole-body miTNM staging system described in Siefert et al., “Second Version of the Prostate Cancer Molecular Imaging Standardized Evaluation Framework Including Response Evaluation for Clinical Trials (PROMISE V2),” Eur Urol. 2023 May; 83(5):405-412. doi: 10.1016/j.eururo.2023.02.002. The notation miTx, for the miT (local tumor) sub-type uses “x” as a placeholder for various alpha numeric codes used in the miTNM system to indicate, for example, whether the local tumor is unifocal or multifocal, whether it is organ-confined or has invaded structures such as the seminal vesicle(s), other adjacent structures such as the external sphincter, rectum, bladder, levator muscles, pelvic wall, and whether it represents a local recurrence after radical prostatectomy. In certain embodiments, such finely-grained information may not be computed, for example due to particular imaging parameters and/or particular anatomical structures segmented. In certain embodiments, additional, finely-grained numeric (e.g., miT2, miT3, miT4) and alphanumeric (e.g., miT2u, miT2m, miT3a, miT3b, miT4, miTr) coding may be computed (e.g., automatically, based on automated anatomical segmentation) and displayed. In certain embodiments, such coding may be computed, but not displayed (e.g., intentionally) in a report such as 400 for sake of simplicity/readability of the report (e.g., to avoid overloading the physician or radiologist). Where level of detail in information, such as detailed miTNM (or other staging systems) coding information, displayed in a high-level report may be limited (e.g., intentionally), systems and methods described herein may include features for providing additional detail. For example, in providing a report such as report 400 via a graphical user interface, a user may be provided with the option to view additional coding information, for example by clicking (or tapping, e.g., in a touch screen device) on or hovering a mouse over portions of report 400. For example, a click or touch interaction may be used to expand summary table 402, allowing for a larger view where additional coding information can be presented, or a click on a particular code, such as “miTx” may be used to bring up (e.g., via a pop-up) additional information.

Generated reports, such as report 400, may also include information such as reference values (e.g., SUV uptake) 404 determined for various reference organs, such as a blood pool (e.g., computed from an aorta region or portion thereof) and a liver, which quantify physiological uptake within the patient, a disease stage code 406, such as an alphanumeric code based on the miTNM scheme, or other schemes. In certain embodiments, disease stage representation 406 includes an indication of the particular staging criteria used. For example, as shown in FIG. 4A, disease stage representation 406 includes the text “miTNM” to indicate use of miTNM staging criteria, along with the particular code determined via analysis of the particular scan(s) on which report 400 is based.

A report may include, additionally or alternatively, a hotspot table 410 that provides a list of each individual hotspot identified, with, for each hotspot, information such as a lesion sub-type, a lesion location (e.g., particular tissue volume in which the lesion is located), and values of various individual hotspot quantification metrics as described herein.

A report as shown in FIG. 4A may, accordingly, be generated from a single imaging session (e.g., a functional and anatomical image, such as a PET/CT or SPECT/CT image) and be used to provide a snapshot of a patient's disease at a particular time.

F. Correlating Patient Indices with Biochemical Progression Free Survival

In certain embodiments, patient risk indices computed from medical images as described herein may be correlated with clinical outcomes, such as biochemical progression free survival (bPFS). As used herein, progression-free survival (PFS), refers to a time as measured from (i) a particular reference point, such as initial diagnosis, one or more treatment date(s), date(s) of particular surveillance checks, random assignment in a clinical trial, etc., to (ii) disease progression or death (e.g., from any cause). In certain embodiments, bPFS refers to a form of PFS whereby disease progression is measured via one or more biochemical markers, such as prostate specific antigen (PSA). For example, in certain embodiments, bPFS may be measured using PSA levels from a subject, with disease progression events determined as PSA values moving outside a particular window or crossing one or more particular threshold values. For example, in certain embodiments, a PSA threshold value of 4.0 ng/mL may be used, with PSA values below 4.0 ng/mL considered normal and PSA values above 4.0 ng/mL used to identify disease progression events.

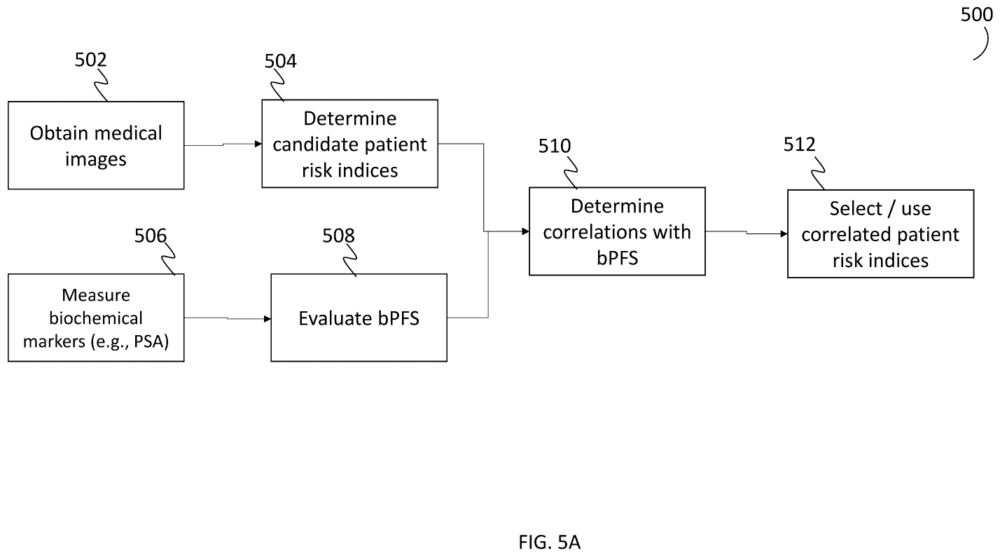

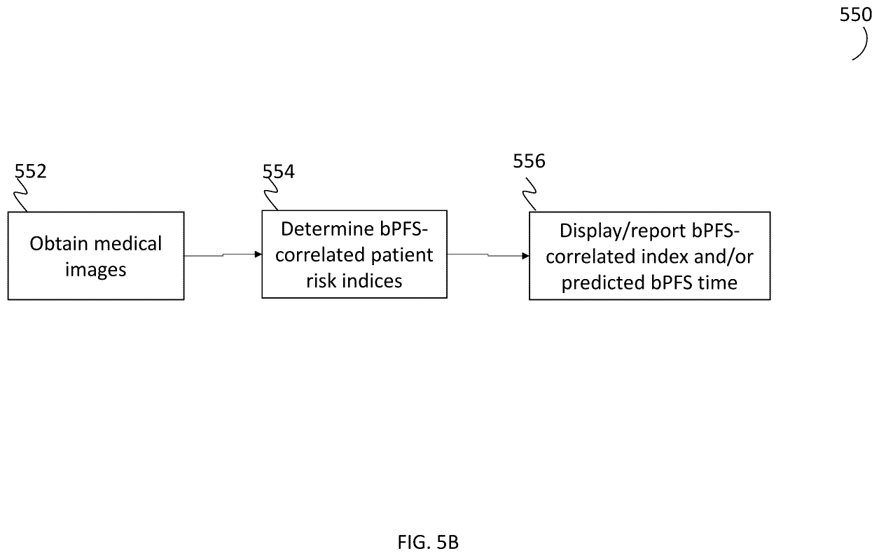

FIG. 5A shows an example process 500 whereby patient risk indices that correlate with bPFS can be identified and then, e.g., later, used and/or displayed in decision support systems and/or automatically or semi-automatically generated reports. As shown in FIG. 5A, medical images may be obtained 502 and analyzed, e.g., as described herein, to determine one or more candidate patient risk indices 504. In certain embodiments, subjects from whom the one or more medical images are obtained may be evaluated, e.g., to measure one or more biochemical markers, such as PSA values 506, and assessed for biochemical progression events 508. In certain embodiments, accordingly, this data can be used to determine correlations between at least a portion of the one or more patient risk indices and bPFS 510. Based on the determined correlations, a subset, such as those that exhibit a high (e.g., positive, or negative) correlation with bPFS may be selected and/or used, e.g., in reporting approaches 512.

For example, as shown example process 550 in FIG. 5B, e.g., once identified, bPFS-correlated patient risk indices can be determined 554 from new medical images (e.g., for a same and/or other, e.g., new, patients) 552 and displayed or otherwise reported 556. In certain embodiments, risk indices themselves may be displayed or reported. In certain embodiments, risk indices may be used to generate an estimate of bPFS (e.g., a time estimate of biochemical progression free survival, e.g., a number of days, weeks, months, etc.) which can be reported.

### G. Computer System and Network Architecture

Certain embodiments described herein make use of computer algorithms in the form of software instructions executed by a computer processor. In certain embodiments, the software instructions include a machine learning module, also referred to herein as artificial intelligence software. As used herein, a machine learning module refers to a computer implemented process (e.g., a software function) that implements one or more specific machine learning techniques, e.g., artificial neural networks (ANNs), e.g., convolutional neural networks (CNNs), e.g., recursive neural networks, e.g., recurrent neural networks such as long short-term memory (LSTM) or Bilateral long short-term memory (Bi-LSTM), random forest, decision trees, support vector machines, and the like, in order to determine, for a given input, one or more output values.

In certain embodiments, machine learning modules implementing machine learning techniques are trained, for example using datasets that include categories of data described herein (e.g., CT images, MRI images, PET images, SPECT images). Such training may be used to determine various parameters of machine learning algorithms implemented by a machine learning module, such as weights associated with layers in neural networks. In certain embodiments, once a machine learning module is trained, e.g., to accomplish a specific task such as segmenting anatomical regions, segmenting and/or classifying hotspots, or determining values for prognostic, treatment response, and/or predictive metrics, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In certain embodiments, machine learning modules may receive feedback, e.g., based on user review of accuracy, and such feedback may be used as additional training data, to dynamically update the machine learning module. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of an ANN module may be carried out via specialized hardware (e.g., via an application specific integrated circuit (ASIC)).

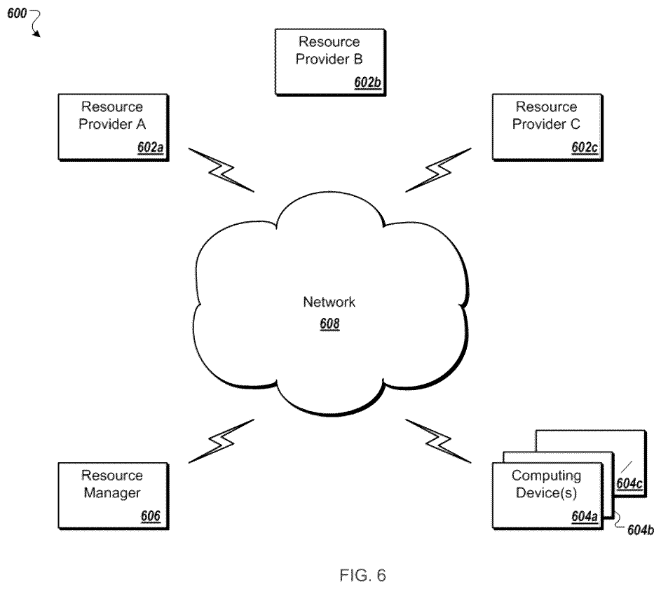

As shown in FIG. 6, an implementation of a network environment 600 for use in providing systems, methods, and architectures as described herein is shown and described. In brief overview, referring now to FIG. 6, a block diagram of an exemplary cloud computing environment 600 is shown and described. The cloud computing environment 600 may include one or more resource providers 602a, 602b, 602c (collectively, 602). Each resource provider 602 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 602 may be connected to any other resource provider 602 in the cloud computing environment 600. In some implementations, the resource providers 602 may be connected over a computer network 608. Each resource provider 602 may be connected to one or more computing device 604a, 604b, 604c (collectively, 604), over the computer network 608.

The cloud computing environment 600 may include a resource manager 606. The resource manager 606 may be connected to the resource providers 602 and the computing devices 604 over the computer network 608. In some implementations, the resource manager 606 may facilitate the provision of computing resources by one or more resource providers 602 to one or more computing devices 604. The resource manager 606 may receive a request for a computing resource from a particular computing device 604. The resource manager 606 may identify one or more resource providers 602 capable of providing the computing resource requested by the computing device 604. The resource manager 606 may select a resource provider 602 to provide the computing resource. The resource manager 606 may facilitate a connection between the resource provider 602 and a particular computing device 604. In some implementations, the resource manager 606 may establish a connection between a particular resource provider 602 and a particular computing device 604. In some implementations, the resource manager 606 may redirect a particular computing device 604 to a particular resource provider 602 with the requested computing resource.

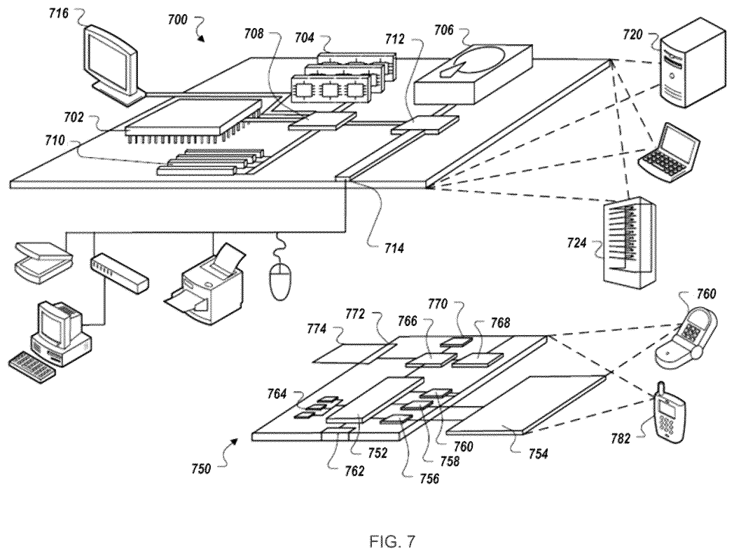

FIG. 7 shows an example of a computing device 700 and a mobile computing device 750 that can be used to implement the techniques described in this disclosure. The computing device 700 is intended to represent various forms of digital computers, such as laptops, desktops, workstations, personal digital assistants, servers, blade servers, mainframes, and other appropriate computers. The mobile computing device 750 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 700 includes a processor 702, a memory 704, a storage device 706, a high-speed interface 708 connecting to the memory 704 and multiple high-speed expansion ports 710, and a low-speed interface 712 connecting to a low-speed expansion port 714 and the storage device 706. Each of the processor 702, the memory 704, the storage device 706, the high-speed interface 708, the high-speed expansion ports 710, and the low-speed interface 712, are interconnected using various busses, and may be mounted on a common motherboard or in other manners as appropriate. The processor 702 can process instructions for execution within the computing device 700, including instructions stored in the memory 704 or on the storage device 706 to display graphical information for a GUI on an external input/output device, such as a display 716 coupled to the high-speed interface 708. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 704 stores information within the computing device 700. In some implementations, the memory 704 is a volatile memory unit or units. In some implementations, the memory 704 is a non-volatile memory unit or units. The memory 704 may also be another form of computer-readable medium, such as a magnetic or optical disk.

The storage device 706 is capable of providing mass storage for the computing device 700. In some implementations, the storage device 706 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 702), perform one or more methods, such as those described above. The instructions can also be stored by one or more storage devices such as computer- or machine-readable mediums (for example, the memory 704, the storage device 706, or memory on the processor 702).

The high-speed interface 708 manages bandwidth-intensive operations for the computing device 700, while the low-speed interface 712 manages lower bandwidth-intensive operations. Such allocation of functions is an example only. In some implementations, the high-speed interface 708 is coupled to the memory 704, the display 716 (e.g., through a graphics processor or accelerator), and to the high-speed expansion ports 710, which may accept various expansion cards (not shown). In the implementation, the low-speed interface 712 is coupled to the storage device 706 and the low-speed expansion port 714. The low-speed expansion port 714, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 700 may be implemented in a number of different forms, as shown in the figure. For example, it may be implemented as a standard server 720, or multiple times in a group of such servers. In addition, it may be implemented in a personal computer such as a laptop computer 722. It may also be implemented as part of a rack server system 724. Alternatively, components from the computing device 700 may be combined with other components in a mobile device (not shown), such as a mobile computing device 750. Each of such devices may contain one or more of the computing device 700 and the mobile computing device 750, and an entire system may be made up of multiple computing devices communicating with each other.

The mobile computing device 750 includes a processor 752, a memory 764, an input/output device such as a display 754, a communication interface 766, and a transceiver 768, among other components. The mobile computing device 750 may also be provided with a storage device, such as a micro-drive or other device, to provide additional storage. Each of the processor 752, the memory 764, the display 754, the communication interface 766, and the transceiver 768, are interconnected using various buses, and several of the components may be mounted on a common motherboard or in other manners as appropriate.

The processor 752 can execute instructions within the mobile computing device 750, including instructions stored in the memory 764. The processor 752 may be implemented as a chipset of chips that include separate and multiple analog and digital processors. The processor 752 may provide, for example, for coordination of the other components of the mobile computing device 750, such as control of user interfaces, applications run by the mobile computing device 750, and wireless communication by the mobile computing device 750.

The processor 752 may communicate with a user through a control interface 758 and a display interface 756 coupled to the display 754. The display 754 may be, for example, a TFT (Thin-Film-Transistor Liquid Crystal Display) display or an OLED (Organic Light Emitting Diode) display, or other appropriate display technology. The display interface 756 may comprise appropriate circuitry for driving the display 754 to present graphical and other information to a user. The control interface 758 may receive commands from a user and convert them for submission to the processor 752. In addition, an external interface 762 may provide communication with the processor 752, so as to enable near area communication of the mobile computing device 750 with other devices. The external interface 762 may provide, for example, for wired communication in some implementations, or for wireless communication in other implementations, and multiple interfaces may also be used.

The memory 764 stores information within the mobile computing device 750. The memory 764 can be implemented as one or more of a computer-readable medium or media, a volatile memory unit or units, or a non-volatile memory unit or units. An expansion memory 774 may also be provided and connected to the mobile computing device 750 through an expansion interface 772, which may include, for example, a SIMM (Single In Line Memory Module) card interface. The expansion memory 774 may provide extra storage space for the mobile computing device 750, or may also store applications or other information for the mobile computing device 750. Specifically, the expansion memory 774 may include instructions to carry out or supplement the processes described above, and may include secure information also. Thus, for example, the expansion memory 774 may be provide as a security module for the mobile computing device 750, and may be programmed with instructions that permit secure use of the mobile computing device 750. In addition, secure applications may be provided via the SIM cards, along with additional information, such as placing identifying information on the SIM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 752), perform one or more methods, such as those described above. The instructions can also be stored by one or more storage devices, such as one or more computer- or machine-readable mediums (for example, the memory 764, the expansion memory 774, or memory on the processor 752). In some implementations, the instructions can be received in a propagated signal, for example, over the transceiver 768 or the external interface 762.

The mobile computing device 750 may communicate wirelessly through the communication interface 766, which may include digital signal processing circuitry where necessary. The communication interface 766 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 768 using a radio-frequency. In addition, short-range communication may occur, such as using a Bluetooth®, Wi-Fi™, or other such transceiver (not shown). In addition, a GPS (Global Positioning System) receiver module 770 may provide additional navigation- and location-related wireless data to the mobile computing device 750, which may be used as appropriate by applications running on the mobile computing device 750.

The mobile computing device 750 may also communicate audibly using an audio codec 760, which may receive spoken information from a user and convert it to usable digital information. The audio codec 760 may likewise generate audible sound for a user, such as through a speaker, e.g., in a handset of the mobile computing device 750. Such sound may include sound from voice telephone calls, may include recorded sound (e.g., voice messages, music files, etc.) and may also include sound generated by applications operating on the mobile computing device 750.

The mobile computing device 750 may be implemented in a number of different forms, as shown in the figure. For example, it may be implemented as a cellular telephone 780. It may also be implemented as part of a smart-phone 782, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, various modules described herein can be separated, combined or incorporated into single or combined modules. Modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

### H. Imaging Agents

As described herein, a variety of radionuclide labelled PSMA binding agents may be used as radiopharmaceutical imaging agents for nuclear medicine imaging to detect and evaluate prostate cancer. In certain embodiments, certain radionuclide labelled PSMA binding agents are appropriate for PET imaging, while others are suited for SPECT imaging.

H.i. PET Imaging Radionuclide Labelled PSMA Binding Agents

In certain embodiments, a radionuclide labelled PSMA binding agent is a radionuclide labelled PSMA binding agent appropriate for PET imaging.

In certain embodiments, a radionuclide labelled PSMA binding agent comprises [18F]DCFPyL (also referred to as PyL™; also referred to as DCFPyL-18F):

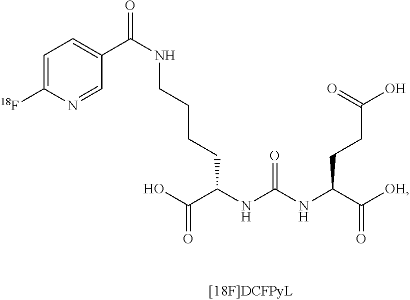 [SMILES: O=C(O)CC[C@H](NC(=O)N[C@@H](CCCCNC(=O)c1ccc([18F])nc1)C(=O)O)C(=O)O]

or a pharmaceutically acceptable salt thereof.

In certain embodiments, a radionuclide labelled PSMA binding agent comprises [18F]DCFBC:

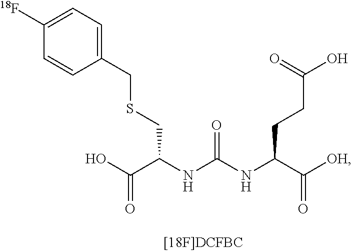 [SMILES: O=C(O)CC[C@H](NC(=O)N[C@@H](CSCc1ccc([18F])cc1)C(=O)O)C(=O)O]

or a pharmaceutically acceptable salt thereof.

In certain embodiments, a radionuclide labelled PSMA binding agent comprises ⁶⁸Ga-PSMA-HBED-CC (also referred to as ⁶⁸Ga-PSMA-11):

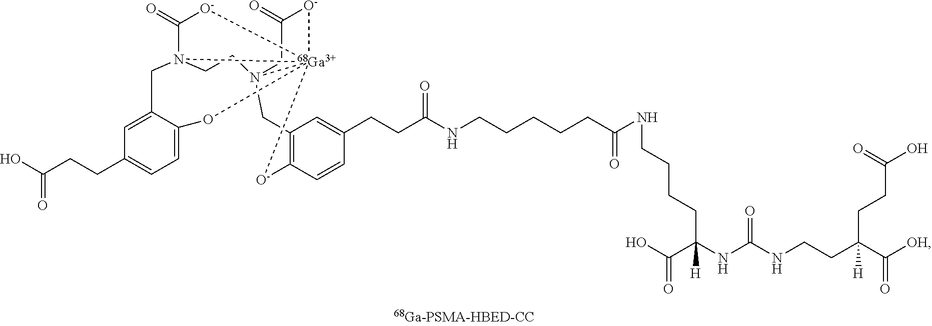

or a pharmaceutically acceptable salt thereof.

In certain embodiments, a radionuclide labelled PSMA binding agent comprises PSMA-617:

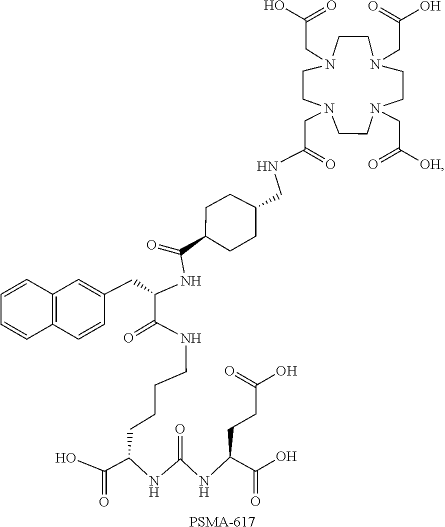

or a pharmaceutically acceptable salt thereof. In certain embodiments, the radionuclide labelled PSMA binding agent comprises ⁶⁸Ga-PSMA-617, which is PSMA-617 labelled with ⁶⁸Ga, or a pharmaceutically acceptable salt thereof. In certain embodiments, a radionuclide labelled PSMA binding agent comprises ¹⁷⁷Lu-PSMA-617, which is PSMA-617 labelled with ¹⁷⁷Lu, or a pharmaceutically acceptable salt thereof.

In certain embodiments, a radionuclide labelled PSMA binding agent comprises PSMA-I&T:

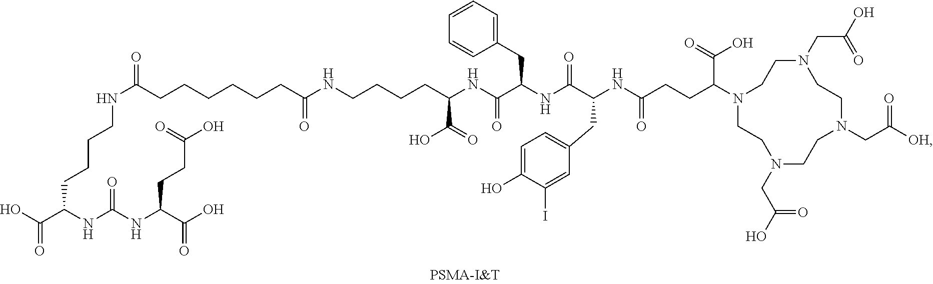 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

or a pharmaceutically acceptable salt thereof. In certain embodiments, a radionuclide labelled PSMA binding agent comprises ⁶⁸Ga-PSMA-I&T, which is PSMA-I&T labelled with ⁶⁸Ga, or a pharmaceutically acceptable salt thereof.

In certain embodiments, a radionuclide labelled PSMA binding agent comprises PSMA-1007:

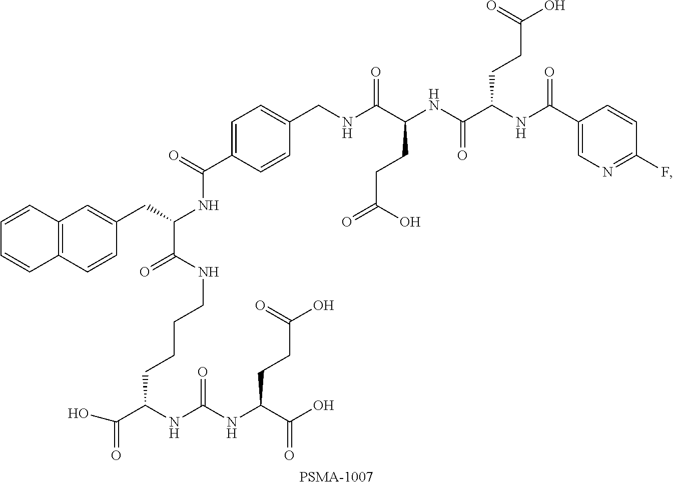

or a pharmaceutically acceptable salt thereof. In certain embodiments, a radionuclide labelled PSMA binding agent comprises ¹⁸F-PSMA-1007, which is PSMA-1007 labelled with ¹⁸F, or a pharmaceutically acceptable salt thereof.

In certain embodiments, a radionuclide labeled PSMA binding agent comprises 18F-JK-PSMA-7:

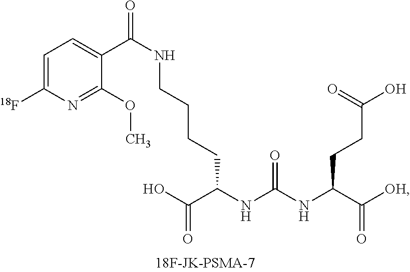 [SMILES: COc1nc([18F])ccc1C(=O)NCCCC[C@H](NC(=O)N[C@@H](CCC(=O)O)C(=O)O)C(=O)O]

or a pharmaceutically acceptable salt thereof.

In certain embodiments, a radionuclide labeled PSMA binding agent comprises (18F) rhPSMA-7.3 (e.g., POSLUMA®, also described at https://www.posluma.com/prescribing-information.pdf):

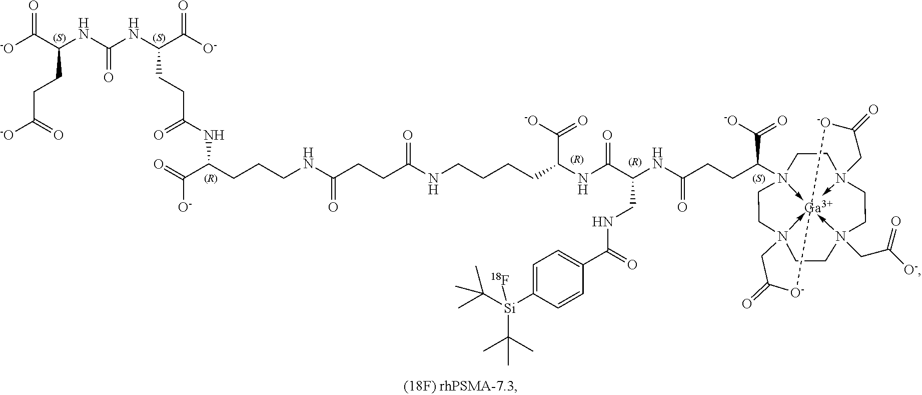

or a pharmaceutically acceptable salt thereof.
H.ii. SPECT Imaging Radionuclide Labelled PSMA Binding Agents

In certain embodiments, a radionuclide labelled PSMA binding agent is a radionuclide labelled PSMA binding agent appropriate for SPECT imaging.

In certain embodiments, a radionuclide labelled PSMA binding agent comprises 1404 (also referred to as MIP-1404):

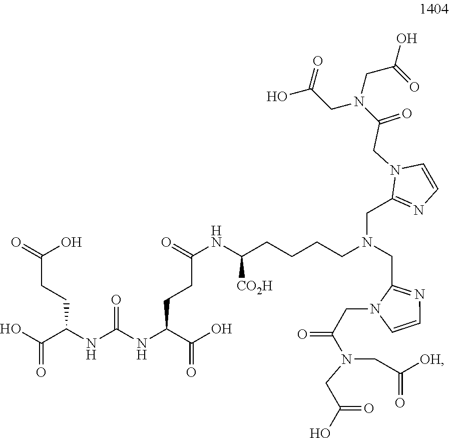 [SMILES: O=C(O)CC[C@H](NC(=O)N[C@@H](CCC(=O)N[C@@H](CCCCN(Cc1nccn1CC(=O)N(CC(=O)O)CC(=O)O)Cc1nccn1CC(=O)N(CC(=O)O)CC(=O)O)C(=O)O)C(=O)O)C(=O)O]

or a pharmaceutically acceptable salt thereof.

In certain embodiments, a radionuclide labelled PSMA binding agent comprises 1405 (also referred to as MIP-1405):

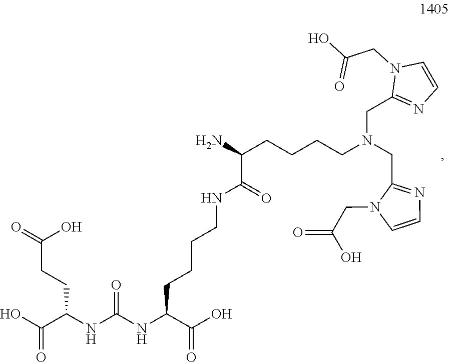 [SMILES: N[C@@H](CCCCN(Cc1nccn1CC(=O)O)Cc1nccn1CC(=O)O)C(=O)NCCCC[C@H](NC(=O)N[C@@H](CCC(=O)O)C(=O)O)C(=O)O]

or a pharmaceutically acceptable salt thereof.

In certain embodiments, a radionuclide labelled PSMA binding agent comprises 1427 (also referred to as MIP-1427):

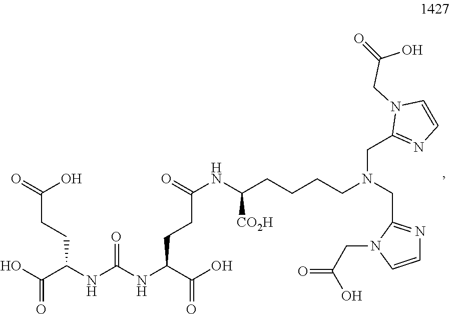 [SMILES: O=C(O)CC[C@H](NC(=O)N[C@@H](CCC(=O)N[C@@H](CCCCN(Cc1nccn1CC(=O)O)Cc1nccn1CC(=O)O)C(=O)O)C(=O)O)C(=O)O]

or a pharmaceutically acceptable salt thereof.

In certain embodiments, a radionuclide labelled PSMA binding agent comprises 1428 (also referred to as MIP-1428):

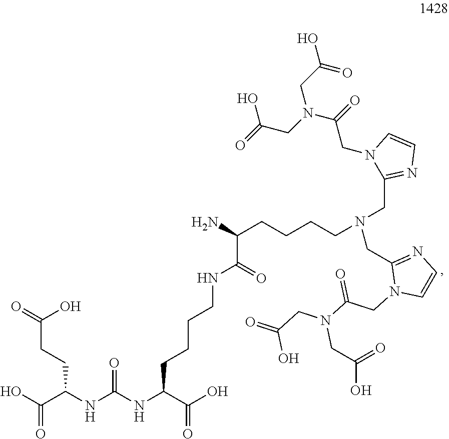 [SMILES: N[C@@H](CCCCN(Cc1nccn1CC(=O)N(CC(=O)O)CC(=O)O)Cc1nccn1CC(=O)N(CC(=O)O)CC(=O)O)C(=O)NCCCC[C@H](NC(=O)N[C@@H](CCC(=O)O)C(=O)O)C(=O)O]

or a pharmaceutically acceptable salt thereof.

In certain embodiments, a PSMA binding agent is labelled with a radionuclide by chelating it to a radioisotope of a metal [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., 68Ga; e.g., 67Ga); e.g., a radioisotope of indium (e.g., 111 In); e.g., a radioisotope of copper (Cu) (e.g., 67Cu)].

In certain embodiments, 1404 is labelled with a radionuclide (e.g., chelated to a radioisotope of a metal). In certain embodiments, a radionuclide labelled PSMA binding agent comprises ^{99m}Tc-MIP-1404, which is 1404 labelled with (e.g., chelated to)^{99m}Tc:

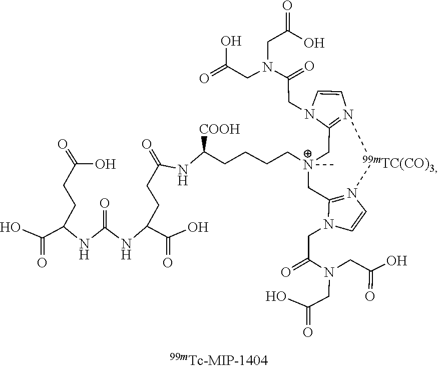

or a pharmaceutically acceptable salt thereof. In certain embodiments, 1404 may be chelated to other metal radioisotopes [e.g., a radioisotope of rhenium (Re) (e.g., rhenium-188 (¹⁸⁸ Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] to form a compound having a structure similar to the structure shown above for ^{99m}Tc-MIP-1404, with the other metal radioisotope substituted for ^{99m}Tc.

In certain embodiments, 1405 is labelled with a radionuclide (e.g., chelated to a radioisotope of a metal). In certain embodiments, a radionuclide labelled PSMA binding agent comprises ^{99m}Tc-MIP-1405, which is 1405 labelled with (e.g., chelated to)^{99m}Tc:

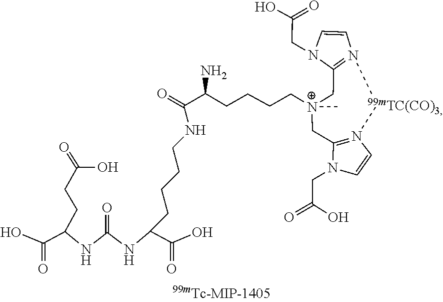

or a pharmaceutically acceptable salt thereof. In certain embodiments, 1405 may be chelated to other metal radioisotopes [e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] to form a compound having a structure similar to the structure shown above for ^{99m}Tc-MIP-1405, with the other metal radioisotope substituted for ^{99m}Tc.

In certain embodiments, 1427 is labelled with (e.g., chelated to) a radioisotope of a metal, to form a compound according to the formula below:

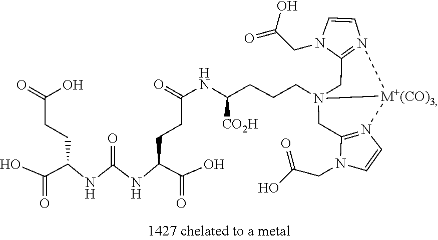

or a pharmaceutically acceptable salt thereof, wherein M is a metal radioisotope [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] with which 1427 is labelled.

In certain embodiments, 1428 is labelled with (e.g., chelated to) a radioisotope of a metal, to form a compound according to the formula below:

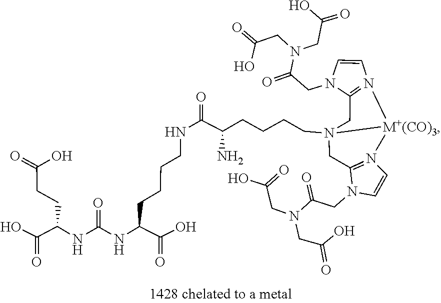

or a pharmaceutically acceptable salt thereof, wherein M is a metal radioisotope [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] with which 1428 is labelled.

In certain embodiments, a radionuclide labelled PSMA binding agent comprises PSMA I&S:

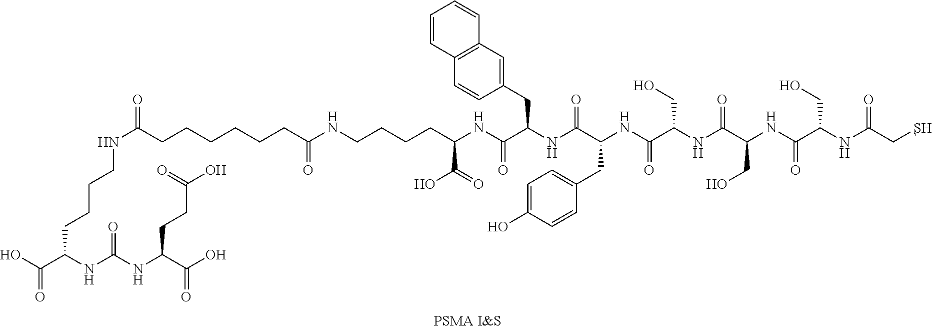 [SMILES: O=C(O)CC[C@H](NC(=O)N[C@@H](CCCCNC(=O)CCCCCCC(=O)NCCCC[C@@H](NC(=O)[C@@H](Cc1ccc2ccccc2c1)NC(=O)[C@@H](Cc1ccc(O)cc1)NC(=O)[C@H](CO)NC(=O)[C@H](CO)NC(=O)[C@H](CO)NC(=O)CS)C(=O)O)C(=O)O)C(=O)O]

or a pharmaceutically acceptable salt thereof. In certain embodiments, a radionuclide labelled PSMA binding agent comprises ^{99m}Tc-PSMA I&S, which is PSMA I&S labelled with ^{99m}Tc, or a pharmaceutically acceptable salt thereof.

### I. EXAMPLES

I.i. Total and Anatomically Contextualized Quantitative ¹⁸F-DCFPyL PET at Biochemical Recurrence Predicts Subsequent Biochemical Progression Free Survival in Prostate Cancer Patients

As described herein, PSMA PET can be used in restaging of prostate cancer at biochemical recurrence. This example describes an experiment demonstrating and assesses clinical utility of quantitative parameters on ¹⁸F-DCFPyL PET/CT at biochemical recurrence and their association with subsequent biochemical progression free survival (bPFS), in accordance with various embodiments described herein.

This example describes a retrospective image analysis and longitudinal follow up of a prospective study evaluating ¹⁸F-DCFPyL PET in prostate cancer patients with biochemical recurrence after primary definitive treatment. The ¹⁸F-DCFPyL PET images were quantitatively analyzed automatically by the aPROMISE application, a semi-automated analysis and structured reporting of PSMA PET/CT. aPROMISE uses deep learning to segment detailed anatomical information from the CT images and uses this information in combination with the PET image to detect and quantify candidates for prostate cancer lesions. The reader works in tandem with the software to vet the final list of lesions, from which the quantitative assessments and final report is created automatically.

Based on the Prostate Cancer Molecular Imaging Standardized Evaluation (PROMISE) criteria, values of the following patient risk indices were obtained based on the miTNM classification: SUVₘₑₐₙ, SUVₘₐₓ, PSMA positive total tumor volume (PSMAₜₜᵥ) and aPSMA scores, a quantitative score for tumor burden measuring the interaction of tumor volume and uptake stratified by local tumors (aPSMA-miT), regional lymph nodes (aPSMA-miN) and distant metastases (aPSMA-miMa for extrapelvic metastases, miMb for bone metastases and miMc for other organ metastases). The association of these quantitative parameters with the subsequent bPFS was evaluated.

Results. One hundred and thirty-four (134) patients (age 70.1±7.6, range 51-91 years; PSA 13.9±98.5, range 0.12-1126 ng/mL) were included in the quantitative image analysis. aPROMISE detected nodal and/or bone metastases in 94 of 134 patients (70%), and only 12 of 134 patients (9%) had visceral disease. With median follow up of 37 months, 66 of 134 patients (49%) had progressed again biochemically after treatment. The bPFS was 25.8±15.1 months. Quantitative analysis of ¹⁸F-DCFPyL PET found that the subsequent bPFS is significantly associated with aPSMA-miMb (P<0.001), PSMAttv (P<0.001), aPSMA-miN (P<0.01), aPSMA-miMa (P<0.05) but not with SUVmean or aPSMA-miMc.

Total and anatomically contextualized quantitative image analysis of ¹⁸F-DCFPyL PET/CT at biochemical recurrence are shown to be useful in predicting the subsequent bPFS.

### EQUIVALENTS

It is to be understood that while the disclosure has been described in conjunction with the detailed description thereof, the foregoing description is intended to illustrate and not limit the scope of the claims. Other aspects, advantages, and modifications are within the scope of the claims.

This written description uses examples to disclose the invention, including the best mode, and also to enable any person skilled in the art to practice the present embodiments, including making and using any devices or systems and performing any incorporated methods. The patentable scope of the present embodiments is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

1. A method for processing one or more images of a prostate cancer patient to automatically determine a patient risk index that correlates with biochemical progression free survival (bPFS) in the patient, the method comprising, the method comprising:
(a) receiving, by a processor of a computing device, an image of the subject obtained using a functional imaging modality; and
(b) identifying, by the processor, one or more patient risk index/indices that correlate with bPFS in the patient.

2. The method of claim 1, wherein the image of the subject is or comprises a 3D PET/CT image.

3. The method of claim 1, wherein the identifying step comprises using deep learning to segment anatomical information from the CT image and use the anatomical information in combination with the PET image to detect and quantify candidates for prostate cancer lesions.

4. The method of claim 1, wherein the one or more determined patient risk index/indices that correlate with bPFS in the patient include one or more members selected from the group consisting of (i) SUVₘₑₐₙ, (ii) SUVₘₐₓ, (iii) PSMA positive total tumor volume (PSMAₜₜᵥ), and (iv) aPSMA scores.

5. The method of claim 4, wherein the PSMAₜₜᵥ is a measure of a total lesion volume for the subject and/or over a subset of lesions within one or more tissue regions and/or prostate cancer staging classes.

6. The method of claim 4, wherein the aPSMA score is a quantitative score for tumor burden measuring the interaction of tumor volume and uptake.

7. The method of claim 1, wherein step (b) comprises:
detecting, by the processor, one or more hotspots within the functional image, each hotspot determined to represent a potential underlying lesion; and
determining, by the processor, the one or more patient risk indices based on the one or more detected hotspots.

8. The method of claim 7, wherein detecting the one or more hotspots comprises using a deep learning model.

9. The method of claim 1, wherein the image of the subject is a nuclear medicine image obtained following administration to the subject of a PSMA binding agent.

10. The method of claim 9, wherein the PSMA binding agent is or comprises [18F]DCFPyL (PyL).

11. The method of claim 1 comprising causing, by the processor, display of the one or more patient risk indices within a graphical user interface (GUI).

12. The method of claim 1, wherein the processor is a processor of a cloud-based system.

13. A system for processing one or more images of a prostate cancer patient to automatically determine a patient risk index that correlates with biochemical progression free survival (bPFS) in the patient, the system comprising:
a processor of a computing device; and
memory having instructions stored thereon, wherein the instructions, when executed by the processor, cause the processor to:
(a) receive an image of the subject obtained using a functional imaging modality; and
(b) identify one or more patient risk index/indices that correlate with bPFS in the patient.

14. The system of claim 13, wherein the image of the subject is or comprises a 3D PET/CT image.

15. The system of claim 13, wherein, at step (b), the instructions cause the processor to identify the one or more patient risk index/indices by using deep learning to segment anatomical information from the CT image and use the anatomical information in combination with the PET image to detect and quantify candidates for prostate cancer lesions.

16. The system of claim 13, wherein the one or more determined patient risk index/indices that correlate with bPFS in the patient include one or more members selected from the group consisting of (i) SUVₘₑₐₙ, (ii) SUVₘₐₓ, (iii) PSMA positive total tumor volume (PSMAₜₜᵥ), and (iv) aPSMA scores.

17. The system of claim 16, wherein the PSMAₜₜᵥ is a measure of a total lesion volume for the subject and/or over a subset of lesions within one or more tissue regions and/or prostate cancer staging classes.

18. The system of claim 16, wherein the aPSMA score is a quantitative score for tumor burden measuring the interaction of tumor volume and uptake.

19. The system of claim 13, wherein, at step (b), the instructions cause the processor to:
detect one or more hotspots within the functional image, each hotspot determined to represent a potential underlying lesion; and
determine the one or more patient risk indices based on the one or more detected hotspots.

20. The system of claim 19, wherein the instructions cause the processor to detect the one or more hotspots using a deep learning model.

21. The system of claim 13, wherein the image of the subject is a nuclear medicine image obtained following administration to the subject of a PSMA binding agent.

22. The system of claim 21, wherein the PSMA binding agent is or comprises [18F]DCFPyL (PyL).

23. The system of claim 13, wherein the instructions cause the processor to cause display of the one or more patient risk indices within a graphical user interface (GUI).

24. The system of claim 13, wherein the system is a cloud-based system.
